(19) United States
(12) Patent Application Publication
(10) Pub. No.: US 2018/0008624 A1
(43) Pub. Date: Jan. 11, 2018

(54) Pharmaceutical Compositions Comprising Ledipasvir And Sofosbuvir

(71) Applicant: Sandoz AG, Basel (CH)

(72) Inventors: Franz Xaver Schwarz, Kundl (AT); Georg ANKER, Kundl (AT); Nolwenn MARTIN, Kundl (AT)

(73) Assignee: Sandoz AG, Basel (CH)

(21) Appl. No.: 15/550,091

(22) PCT Filed: Feb. 10, 2016
(86) PCT No.: PCT/EP2016/052805

§ 371 Date: Aug. 10, 2017

(30) Foreign Application Priority Data
Feb. 13, 2015 (EP) 15155094.4

(51) Int. Cl. A61K 31/7072; A61K 9/20; A61K 31/439

(52) U.S. Cl. CPC A61K 31/7072; A61K 31/439; A61K 9/2027; A61K 9/2054

(57) ABSTRACT

The present invention relates to novel pharmaceutical compositions comprising Ledipasvir and Sofosbuvir as well as to methods for their preparation.

The present invention relates to pharmaceutical compositions comprising Ledipasvir and Sofosbuvir as well as processes for the preparation of such pharmaceutical compositions. Further, the present invention relates to the use of the pharmaceutical compositions comprising Ledipasvir and Sofosbuvir for the treatment of Hepatitis C.

Ledipasvir According to Formula (I)

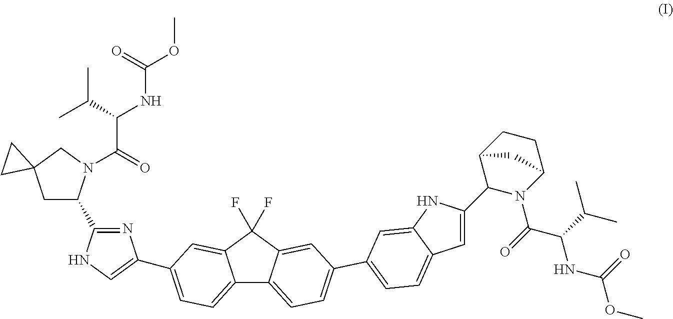 [SMILES: COC(=O)N[C@H](C(=O)N1CC2(CC2)C[C@H]1c1nc(-c2ccc3c(c2)C(F)(F)c2cc(-c4ccc5cc(C6[C@H]7CC[C@H](C7)N6C(=O)[C@@H](NC(=O)OC)C(C)C)[nH]c5c4)ccc2-3)c[nH]1)C(C)C]

with IUPAC name methyl N-[(2S)-1-[(6S)-6-[5-[9,9-difluoro-7-[2-[(1S,2S,4R)-3-[(2S)-2-(methoxycarbonylamino)-3-methylbutanoyl]-3-azabicyclo[2.2.1]heptan-2-yl]-3H-benzimidazol-5-yl]fluoren-2-yl]-1H-imidazol-2-yl]-5-azaspiro[2.4]heptan-5-yl]-3-methyl-1-oxobutan-2-yl]carbamate and Sofosbuvir according to formula (II)

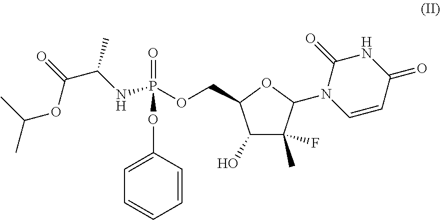 [SMILES: CC(C)OC(=O)[C@H](C)N[P@](=O)(OC[C@H]1OC(n2ccc(=O)[nH]c2=O)[C@](C)(F)[C@@H]1O)Oc1ccccc1]

with IUPAC name (S)-isopropyl 2-(((S)-(((2R,3R,4R,5R)-5-(2,4-dioxo-3,4-dihydropyrimidin-1(2H)-yl)-4-fluoro-3-hydroxy-4-methyltetrahydrofuran-2-yl)methoxy)(phenoxy)phosphoryl)-amino)propanoate are drugs inhibiting the RNA polymerase used by the Hepatitis C virus to replicate its RNA.

WO2014/120981 describes pharmaceutical compositions comprising Ledipasvir in substantially amorphous form and Sofosbuvir in a substantially crystalline form. However, employing these two active compounds in different forms (i.e. amorphous and crystalline) can affect their dissolution profile and therefore their bioavailability. In particular, WO2014/120981 states that “Further, according to conventional wisdom, it is not advisable to co-formulate an amorphous agent with a crystalline agent, because the crystals can serve as seeds to induce crystallization of the amorphous agent, leading to instability of the amorphous agent.”. The pharmaceutical compositions of WO2014/120981 comprise admixing an independently prepared solid dispersion of Ledipasvir with crystalline Sofosbuvir. Thus, a solid dispersion of Ledipasvir has to be prepared first, which is then mixed with crystalline Sofosbuvir to prepare single-layer or bilayer tablets. This requires therefore the independent preparation of a solid dispersion of Ledipasvir, which adds at least one additional step to the process.

Thus, there is a need for the provision of novel pharmaceutical compositions comprising the compound of formula (I) (i.e. Ledipasvir) and the compound of formula (II) (i.e. Sofosbuvir) which show good bioavailability increasing the synergistic effects of both active ingredients and that can be produced industrially in an efficient manner, i.e. which production is cost-effective and does not involve the use of large quantities of organic solvents or of hazardous reagents. Therefore, the problem underlying the present invention is the provision of novel pharmaceutical compositions comprising the compound of formula (I) and the compound of formula (II) as well as the provision of novel and efficient methods for the provision of said novel compositions.

It was surprisingly found that such compositions comprising the compound of formula (I) and the compound of formula (II) as described in the present invention fulfill these requirements and that it is possible to prepare said compositions in an efficient and effective manner.

In particular, it was surprisingly found that compositions of the present invention comprising the compound of formula (I) and the compound of formula (II) in which these two compounds are in amorphous form fulfill the requirements mentioned above and can be prepared in an efficient and effective manner, even though, as stated in WO2014/120981, “ . . . amorphous agents are expected to be unstable and have nonlinear solubility and exposure profiles.”.

1. Pharmaceutical Compositions Comprising the Compound of Formula (I) and the Compound of Formula (II)

The present invention relates to pharmaceutical compositions comprising a compound of formula (I) or a pharmaceutically acceptable salt or solvate thereof and a compound of formula (II) or a pharmaceutically acceptable salt or solvate thereof,

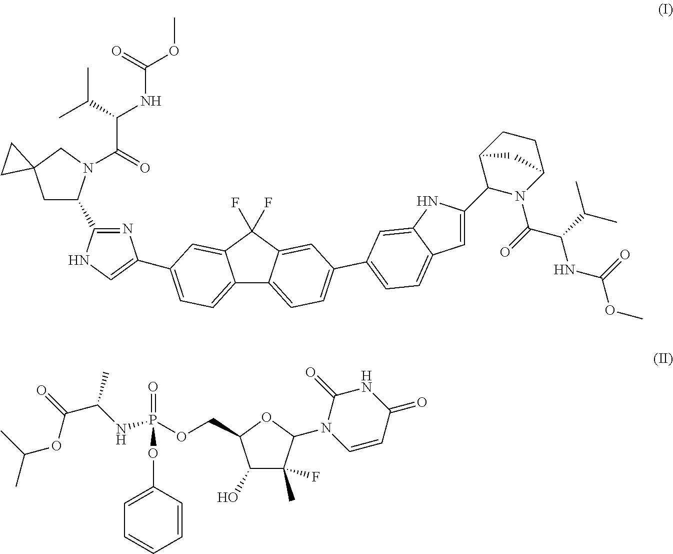

wherein the compound of formula (I) and the compound of formula (II) can be in crystalline or amorphous form, and wherein when the compound of formula (I) is amorphous and the compound of formula (II) is crystalline the compound of formula (II) is the crystalline Form VII, i.e. it has an X-ray powder diffraction pattern comprising no reflection at 2-theta angles in the range of from 2.0 to 7.8°, when measured at a temperature in the range of from 15 to 25° C. with Cu-Kalpha_{1,2} radiation having a wavelength of 0.15419 nm and/or an X-ray powder diffraction pattern comprising reflections at 2-theta values of (8.1±0.2)°, (10.4±0.2)°, (12.4±0.2)°, (17.3±0.2)°, (19.4±0.2)°, when measured at a temperature in the range of from 15 to 25° C. with Cu-Kalpha_{1,2} radiation having a wavelength of 0.15419 nm.

In the context of the present invention, the term “amorphous” refers to a state in which the material lacks long range order at the molecular level and, depending upon temperature, may exhibit the physical properties of a solid or a liquid. Typically such materials do not give distinctive X-ray diffraction patterns and, while exhibiting the properties of a solid, are more formally described as a liquid. Upon heating, a change from solid to liquid properties occurs which is characterized by a change of state, typically second order (glass transition).

In the context of the present invention, the term “crystalline” refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterized by a phase change, typically first order (melting point).

1.1 the Compound of Formula (I) and the Compound of Formula (II)—Amorphous and Crystalline Forms

The pharmaceutical composition of the invention is in one aspect characterized in that it comprises a compound of formula (I), i.e. Ledipasvir, in crystalline or in essentially crystalline form.

The compound of formula (I), i.e. Ledipasvir, has previously been described, for example in WO2010/132601. Crystalline forms of Ledipasvir have been described in WO2013/184698. Crystalline Form I of Ledipasvir is characterized by an X-ray powder diffraction pattern comprising reflections at 2-theta values of (3.4±0.2)°, (6.8±0.2)°, (11.0±0.2)°, (12.5±0.2)° and (19.8±0.2)° when measured with Cu-Kalpha_{1,2} radiation having a wavelength of 1.54178 Å. Crystalline Form II of Ledipasvir is characterized by an X-ray powder diffraction pattern comprising reflections at 2-theta values of (11.4±0.2)°, (12.2±0.2)°, (12.7±0.2)°, (20.0±0.2)° and (20.5±0.2)° when measured with Cu-Kalpha_{1,2} radiation having a wavelength of 1.54178 Å. Crystalline Form III of Ledipasvir is characterized by an X-ray powder diffraction pattern comprising reflections at 2-theta values of (8.3±0.2)°, (12.4±0.2)°, (14.2±0.2)°, (15.0±0.2)° and (21.6±0.2)° when measured with Cu-Kalpha_{1,2} radiation having a wavelength of 1.54178 Å.

Thus preferably, the present invention relates to a pharmaceutical composition wherein the compound of formula (I) is crystalline and is the crystalline Form I, the crystalline Form II, the crystalline Form III or a mixture of two thereof.

Preferably, the present invention also relates to mixtures comprising two or more crystalline forms of Ledipasvir, for example mixtures comprising crystalline forms I and II, mixtures comprising crystalline forms I and III, mixtures comprising crystalline forms II and III and mixtures comprising crystalline forms I, II and III. Mixtures comprising at least two of any of the crystalline forms described in WO2013/184698 are also within the scope of the present invention.

Thus preferably, the present invention relates to a pharmaceutical composition wherein the compound of formula (I) is crystalline and comprises a mixture of two or more crystalline forms selected from the crystalline Form I, the crystalline Form II or the crystalline Form III.

The pharmaceutical composition of the invention is in another aspect characterized in that it comprises a compound of formula (II), i.e. Sofosbuvir, in crystalline or in essentially crystalline form.

Sofosbuvir has previously been described, for example in WO2008/121634. In particular, amorphous Sofosbuvir is described in WO2010/135569 and crystalline forms of Sofosbuvir have been described in WO2010/135569 and WO2011/123645. Specifically, crystalline Form I of Sofosbuvir is characterized by an X-ray powder diffraction pattern comprising reflections at 2-theta values of (5.0±0.2)°, (7.3±0.2)°, (9.4±0.2)°, (17.3±0.2)°, (18.1±0.2)°, when measured at a temperature in the range of from 15 to 25° C. with Cu-Kalpha_{1,2} radiation having a wavelength of 0.15419 nm. Crystalline Form VI of Sofosbuvir is characterized by an X-ray powder diffraction pattern comprising reflections at 2-theta values of (6.1±0.2) °, (8.2±0.2)°, (12.7±0.2)°, (20.1±0.2)°, (20.8±0.2)°. Crystalline Form VII of Sofosbuvir has an X-ray powder diffraction pattern comprising no reflection at 2-theta angles in the range of from 2.0 to 7.8°, when measured at a temperature in the range of from 15 to 25° C. with Cu-Kalpha_{1,2} radiation having a wavelength of 0.15419 nm and/or an X-ray powder diffraction pattern comprising reflections at 2-theta values of (8.1±0.2)°, (10.4±0.2)°, (12.4±0.2)°, (17.3±0.2)°, (19.4±0.2)°, when measured at a temperature in the range of from 15 to 25° C. with Cu-Kalpha_{1,2} radiation having a wavelength of 0.15419 nm. Additionally, Form VII of Sofosbuvir can be characterized by the following embodiments and combination of embodiments as indicated by the respective back-references:

1. A crystalline form of Sofosbuvir of formula (II) (Form 7/VII)

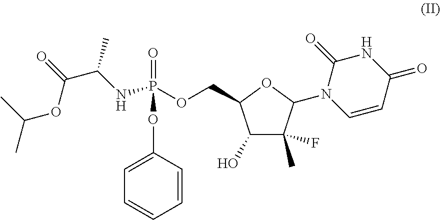 [SMILES: CC(C)OC(=O)[C@H](C)N[P@](=O)(OC[C@H]1OC(n2ccc(=O)[nH]c2=O)[C@](C)(F)[C@@H]1O)Oc1ccccc1]

having an X-ray powder diffraction pattern comprising no reflection at 2-theta angles in the range of from 2.0 to 7.8°, when measured at a temperature in the range of from 15 to 25° C. with Cu-Kalpha1,2 radiation having a wavelength of 0.15419 nm.

2. The crystalline form of embodiment 1, having an X-ray powder diffraction pattern comprising reflections at 2-theta values of (8.1±+0.2)°, (10.4±0.2)°, (12.4±0.2)°, (17.3±0.2)°, (19.4±0.2)°, when measured at a temperature in the range of from 15 to 25° C. with Cu-Kalpha1,2 radiation having a wavelength of 0.15419 nm.

3. The crystalline form of embodiment 1 or 2, exhibiting a Fourier transform infrared spectrum comprising peaks at wavenumbers of (3252±2) cm⁻¹, (2928±2) cm⁻¹, (1718±2) cm⁻¹, (1668±2) cm⁻¹, (1456±2) cm⁻¹, when measured at a temperature in the range of from 15 to 25° C. using a ZnSe ATR cell.

4. The crystalline form of any of embodiments 1 to 3, having the monoclinic space group symmetry P2₁ and the following unit cell parameters as determined by an X-ray single-crystal structure analysis at 120 K:

a=(5.16±0.04) Angstrom;

b=(16.86±0.12) Angstrom;

c=(14.44±0.10) Angstrom;

alpha=90.0°;

beta=(100.2±0.8)°;

gamma=90.0°.

5. The crystalline form of any of embodiments 1 to 4, having a melting point in the range of from 122 to 126° C. when measured via differential scanning calorimetry at a heating rate of 10 K/min at a pressure in the range of from 0.95 to 1.05 bar.

6. The crystalline form of any of embodiments 1 to 5, comprising at most 0.5 weight-% of organic solvent, based on the weight of the crystalline form, as determined via thermogravimetric analysis.

7. The crystalline form of any of embodiments 1 to 6, comprising at most 0.4 weight-% of water based on the weight of the crystalline form as determined via gravimetric moisture sorption/desorption analysis at a temperature of (25.0±0.1)° C. and a relative humidity of from 0 to 95%.

This crystalline Form 7/VII of sofosbuvir is the only crystalline form of sofosbuvir showing no peak at 2-theta angles in the range of from 2 to 7.8° in the XRPD pattern. All other known crystalline forms according to the prior art show at least one significant peak in this range, as summarized in the following Table:

TABLE 1
XRPD peaks of prior art crystalline forms
in the range of from 2 to 7.8° 2-theta

| | Peak Positions/ | |
|---|---|---|
| Prior art forms | ° 2-theta | Prior art document |
| Form 1/I | 5.0, 7.3, 7.8 | table 2 ex WO 2010/135569 A1 |
| Form 2/II | 4.9, 5.1, 6.9 | table 3 ex WO 2010/135569 A1 |
| Form 3/III | 5.0, 6.9 | table 4 ex WO 2010/135569 A1 |
| Form 4/IV | 5.0, 6.8 | table 5 ex WO 2010/135569 A1 |
| Form 5/V | 5.2, 6.6, 7.1 | table 6 ex WO 2010/135569 A1 |
| Form 6/VI | 6.1 | table page 95-96 WO 2011/123645 A1 |

Hence, the absence of an XRPD peak in said range is unique and therefore a characteristic property of this crystalline form of Sofosbuvir. Additionally, this crystalline Form VII of Sofosbuvir can be, for example, further distinguished from crystalline Form 1 of WO 2010/135569 A1 by a characteristic XRPD peak at (12.4±0.2)° 2-theta since the crystalline Form 1 shows no such characteristic peak in this range when measured at room temperature with Cu-Kalpha1,2 radiation having a wavelength of 0.15419 nm.

Preferably, the present invention relates to a pharmaceutical composition wherein the compound of formula (II) is crystalline and is the crystalline Form I, the crystalline Form VI or the crystalline Form VII.

1.1.1 the Compound of Formula (I) and the Compound of Formula (II) are Crystalline

The present invention also relates to the pharmaceutical composition of the invention wherein the compound of formula (I) is crystalline and the compound of formula (II) is crystalline.

Preferably, the compound of formula (I) is crystalline and the compound of formula (II) is crystalline and is the crystalline Form I, the crystalline Form VI or the crystalline Form VII.

Preferably, the compound of formula (I) is crystalline and is the crystalline Form I, the crystalline Form II or the crystalline Form III or a mixture of two or more of these forms and the compound of formula (II) is crystalline.

Preferably, the compound of formula (I) is crystalline and is the crystalline Form I, the crystalline Form II or the crystalline Form III and the compound of formula (II) is crystalline and is the crystalline Form I, the crystalline Form VI or the crystalline Form VII.

Preferably, the compound of formula (I) is crystalline and comprises a mixture of two or more crystalline forms and the compound of formula (II) is crystalline.

The present invention also relates to the pharmaceutical composition of the invention wherein the compound of formula (I) is crystalline and comprises a pure crystalline form or a mixture of two or more crystalline forms selected from the crystalline Form I, the crystalline Form II and the crystalline Form III and the compound of formula (II) is crystalline.

Preferably, the compound of formula (I) is crystalline and comprises a pure crystalline form or a mixture of two or more crystalline forms and the compound of formula (II) is crystalline and is the crystalline Form I, the crystalline Form VI or the crystalline Form VII.

Preferably, the compound of formula (I) is crystalline and comprises a pure crystalline form or a mixture of two or more crystalline forms selected from the crystalline Form I, the crystalline Form II and the crystalline Form III and the compound of formula (II) is crystalline and is the crystalline Form I, the crystalline Form VI or the crystalline Form VII.

Preferably, the compound of formula (I) is crystalline and comprises a pure crystalline form or a mixture of two or more crystalline forms selected from the crystalline Form I, the crystalline Form II and the crystalline Form III and the compound of formula (II) is crystalline and is the crystalline Form VII.

Preferably, the compound of formula (I) is crystalline and is the crystalline Form I and the compound of formula (II) is crystalline and is the crystalline Form VII.

Preferably, the compound of formula (I) is crystalline and is the crystalline Form II and the compound of formula (II) is crystalline and is the crystalline Form VII.

Preferably, the compound of formula (I) is crystalline and is the crystalline Form III and the compound of formula (II) is crystalline and is the crystalline Form VII.

Preferably, the compound of formula (I) is crystalline and comprises a pure crystalline form or a mixture of two or more crystalline forms selected from the crystalline Form I, the crystalline Form II and the crystalline Form III and the compound of formula (II) is crystalline and is the crystalline Form I.

Preferably, the compound of formula (I) is crystalline and is the crystalline Form I and the compound of formula (II) is crystalline and is the crystalline Form I.

Preferably, the compound of formula (I) is crystalline and is the crystalline Form II and the compound of formula (II) is crystalline and is the crystalline Form I.

Preferably, the compound of formula (I) is crystalline and is the crystalline Form III and the compound of formula (II) is crystalline and is the crystalline Form I.

1.1.2 the Compound of Formula (II) is Amorphous

The present invention also relates to the pharmaceutical composition of the invention wherein the compound of formula (I) is crystalline and the compound of formula (II) is amorphous.

Preferably, the compound of formula (I) is crystalline and is the crystalline Form I, the crystalline Form II or the crystalline Form III or a mixture of two or more of these forms and the compound of formula (II) is amorphous.

Preferably, the compound of formula (I) is crystalline and comprises a mixture of two or more crystalline forms and the compound of formula (II) is amorphous.

Preferably, the compound of formula (I) is crystalline and comprises a mixture of two or more crystalline selected from the crystalline Form I, the crystalline Form II or the crystalline Form III and the compound of formula (II) is amorphous.

Preferably, the compound of formula (I) is crystalline and is the crystalline Form I and the compound of formula (II) is amorphous.

Preferably, the compound of formula (I) is crystalline and is the crystalline Form II and the compound of formula (II) is amorphous.

Preferably, the compound of formula (I) is crystalline and is the crystalline Form III and the compound of formula (II) is amorphous.

Preferably, the compound of formula (I) comprises a mixture of the crystalline Form I and of the crystalline Form II and the compound of formula (II) is amorphous.

Preferably, the compound of formula (I) comprises a mixture of the crystalline Form I and of the crystalline Form III and the compound of formula (II) is amorphous.

Preferably, the compound of formula (I) comprises a mixture of the crystalline Form II and of the crystalline Form III and the compound of formula (II) is amorphous.

Preferably, the compound of formula (I) comprises a mixture of the crystalline Form I, the crystalline Form II and the crystalline Form III and the compound of formula (II) is amorphous.

1.1.3 the Compound of Formula (I) and/or the Compound of Formula (II) is/are Amorphous

The present invention also relates to the pharmaceutical composition of the invention wherein the compound of formula (I) is amorphous.

The present invention also relates to the pharmaceutical composition of the invention wherein the compound of formula (II) is amorphous.

Preferably, the compound of formula (I) is amorphous and the compound of formula (II) is amorphous.

The present invention also relates to the pharmaceutical composition of the invention wherein the compound of formula (I) is amorphous and the compound of formula (II) is crystalline and is the crystalline Form VII.

1.2 the Compound of Formula (I) and the Compound of Formula (II)—Solid Dispersions and Melts

Additionally, the present invention relates to any of the compositions described above wherein the compound of formula (I) or the compound of formula (II) is a solid dispersion or a melt.

In the context of the present invention, the term “solid dispersion” relates to a composition in a solid state, i.e. a state which is neither liquid nor gaseous, wherein the compound of formula (I) or the compound of formula (II) is dispersed in at least one pharmaceutically acceptable matrix. The solid dispersions according to the present invention can be prepared by a variety of methods, including spray drying, the melting (fusion), extrusion and solvent evaporation.

Thus, the present invention relates to the pharmaceutical composition of the invention wherein the compound of formula (I) is a solid dispersion. It also relates to the pharmaceutical composition of the invention wherein the compound of formula (II) is a solid dispersion.

Preferably, the compound of formula (I) is an amorphous solid dispersion. Preferably, the compound of formula (II) is an amorphous solid dispersion.

In the context of the present invention, the term “amorphous solid dispersion” as used herein, refers to stable solid dispersions wherein the amorphous compound of the formula (I) or the amorphous compound of the formula (II) is dispersed in at least one pharmaceutically acceptable matrix.

Preferably, the compound of formula (I) is a crystalline solid dispersion. Preferably, the compound of formula (II) is a crystalline solid dispersion.

In the context of the present invention, the term “crystalline solid dispersion” as used herein, refers to stable solid dispersions wherein the compound of the formula (I) or the compound of the formula (II) is dispersed in at least one pharmaceutically acceptable matrix, wherein the compound of the formula (I) or the compound of formula (II) are present in a crystalline state as defined above.

Preferably, the compound of formula (I) or the compound of formula (II) is present in any of the crystalline forms described above, or mixtures thereof. The specific crystalline forms of the compound of formula (I) (i.e. Forms I, II and III of Ledipasvir) and the specific crystalline forms of the compound of formula (II) (i.e. Forms I, VI and VII of Sofosbuvir) are as defined above.

1.2.1 the Compound of Formula (I) and the Compound of Formula (II) are a Solid Dispersion

The present invention also relates to the pharmaceutical composition of the invention wherein the compound of formula (I) and the compound of formula (II) are a solid dispersion.

Preferably, the compound of formula (I) is an amorphous solid dispersion and the compound of formula (II) is an amorphous solid dispersion.

Preferably, the compound of formula (I) is an amorphous solid dispersion and the compound of formula (II) is a crystalline solid dispersion.

Preferably, the compound of formula (I) is an amorphous solid dispersion and the compound of formula (II) is a crystalline solid dispersion comprising the crystalline Form I, the crystalline Form VI or the crystalline Form VII or mixtures thereof.

Preferably, the compound of formula (I) is an amorphous solid dispersion and the compound of formula (II) is a crystalline solid dispersion comprising the crystalline Form VII.

Preferably, the compound of formula (I) is a crystalline solid dispersion and the compound of formula (II) is an amorphous solid dispersion.

Preferably, the compound of formula (I) is a crystalline solid dispersion comprising the crystalline Form I, the crystalline Form II or the crystalline Form III or a mixture of two or more of these forms and the compound of formula (II) is an amorphous solid dispersion.

Preferably, the compound of formula (I) is a crystalline solid dispersion and the compound of formula (II) is a crystalline solid dispersion.

Preferably, the compound of formula (I) is a crystalline solid dispersion comprising the crystalline Form I, the crystalline Form II or the crystalline Form III or a mixture of two or more of these forms and the compound of formula (II) is a crystalline solid dispersion.

Preferably, the compound of formula (I) is a crystalline solid dispersion and the compound of formula (II) is a crystalline solid dispersion comprising the crystalline Form I, the crystalline Form VI or the crystalline Form VII or mixtures thereof.

Preferably, the compound of formula (I) is a crystalline solid dispersion comprising the crystalline Form I, the crystalline Form II or the crystalline Form III or a mixture of two or more of these forms and the compound of formula (II) is a crystalline solid dispersion comprising the crystalline Form I, the crystalline Form VI or the crystalline Form VII or mixtures thereof.

Preferably, the compound of formula (I) is a crystalline solid dispersion comprising the crystalline Form I, the crystalline Form II or the crystalline Form III or a mixture of two or more of these forms and the compound of formula (II) is a crystalline solid dispersion comprising the crystalline Form VII.

Preferably, the compound of formula (I) is a crystalline solid dispersion comprising the crystalline Form I, the crystalline Form II or the crystalline Form III or a mixture of two or more of these forms and the compound of formula (II) is a crystalline solid dispersion comprising the crystalline Form I.

The present invention also relates to the pharmaceutical composition of the invention wherein the compound of formula (I) and the compound of formula (II) are a homogeneous solid dispersion. In the context of the present invention, a homogeneous solid dispersion of the compounds of formula (I) and (II) is to be understood as a solid dispersion as defined above wherein the compound of formula (I) and the compound of formula (II) are dispersed in at least one pharmaceutically acceptable matrix. The compound of formula (I) can be amorphous or crystalline. Preferably, the compound of formula (I) is crystalline and is the crystalline Form I, the crystalline Form II or the crystalline Form III or a mixture of two or more of these forms. The compound of formula (II) can be amorphous or crystalline. Preferably, the compound of formula (I) is crystalline and is the crystalline Form I, the crystalline Form VI or the crystalline Form VII.

Preferably, the compound of formula (I) is amorphous and the compound of formula (II) is amorphous.

Preferably, the compound of formula (I) is amorphous and the compound of formula (II) is crystalline.

Preferably, the compound of formula (I) is amorphous and the compound of formula (II) is crystalline and is the crystalline Form I, the crystalline Form VI or the crystalline Form VII. Preferably, the compound of formula (I) is amorphous and the compound of formula (II) is crystalline and is the crystalline Form VII.

Preferably, the compound of formula (I) is crystalline and the compound of formula (II) is amorphous.

Preferably, the compound of formula (I) is crystalline and is the crystalline Form I, the crystalline Form II or the crystalline Form III or a mixture of two or more of these forms and the compound of formula (II) is amorphous. Preferably, the compound of formula (I) is crystalline and the compound of formula (II) is crystalline. Preferably, the compound of formula (I) is crystalline and the compound of formula (II) is crystalline and is the crystalline Form I, the crystalline Form VI or the crystalline Form VII. Preferably, the compound of formula (I) is crystalline and is the crystalline Form I, the crystalline Form II or the crystalline Form III or a mixture of two or more of these forms and the compound of formula (II) is crystalline.

Preferably, the compound of formula (I) is crystalline and is the crystalline Form I, the crystalline Form II or the crystalline Form III or a mixture of two or more of these forms and the compound of formula (II) is crystalline and is the crystalline Form I, the crystalline Form VI or the crystalline Form VII. Preferably, the compound of formula (I)) is crystalline and is the crystalline Form VII.

1.2.2 the Compound of Formula (II) is a Melt

The present invention also relates to the pharmaceutical composition of the invention wherein the compound of formula (II) is a melt. In the context of the present invention, the term “melt” refers to the product obtained after subjecting the compound of formula (II) or a mixture comprising the compound of formula (II) to a temperature sufficient to completely melt the compound of formula (II) and after cooling said mixture below its melting point, preferably until it completely solidifies.

The amorphous from of the compound of formula (II) has a melting point of 52-56° C. when measured by differential scanning calorimetry at a heating rate of 10 K/min at a pressure in the range of from 0.95 to 1.05 bar. The crystalline Form I of the compound of formula (II) has a melting point of 82-88° C. when measured by differential scanning calorimetry at a heating rate of 10 K/min at a pressure in the range of from 0.95 to 1.05 bar and the crystalline Form VI and the crystalline Form VII of the compound of formula (II) have a melting point of 120-126° C. when measured by differential scanning calorimetry at a heating rate of 10 K/min at a pressure in the range of from 0.95 to 1.05 bar.

Thus, the present invention relates to the pharmaceutical composition of the invention wherein the compound of formula (II) is a melt.

Preferably, the compound of formula (I) is a solid dispersion and the compound of formula (II) is a melt.

Preferably, the compound of formula (I) is an amorphous solid dispersion and the compound of formula (II) is a melt.

Preferably, the compound of formula (I) is a crystalline solid dispersion and the compound of formula (II) is a melt.

Preferably, the compound of formula (I) is a crystalline solid dispersion comprising the crystalline Form I, the crystalline Form II or the crystalline Form III or mixtures thereof and the compound of formula (II) is a melt.

The present invention also relates to the pharmaceutical composition of the invention wherein the compound of formula (II) is a melt further comprising at least one pharmaceutically acceptable matrix.

Preferably, the compound of formula (I) is an amorphous solid dispersion and the compound of formula (II) is a melt further comprising at least one pharmaceutically acceptable matrix.

Preferably, the compound of formula (I) is a crystalline solid dispersion and the compound of formula (II) is a melt further comprising at least one pharmaceutically acceptable matrix.

Preferably, the compound of formula (I) is a crystalline solid dispersion comprising the crystalline Form I, the crystalline Form II or the crystalline Form III or mixtures thereof and the compound of formula (II) is a melt further comprising at least one pharmaceutically acceptable matrix.

1.3 the Compound of Formula (I) and the Compound of Formula (II)—Amorphous and Crystalline Forms in Combination with Solid Dispersions and Melts

The present invention also relates to the pharmaceutical composition of the invention wherein one of the compound of formula (I) or of formula (II) (i.e. the compound of formula (I) or the compound of formula (II)) is a solid dispersion or a melt and the other one is not a solid dispersion or a melt. In particular, the other one is not admixed with any other component such as an excipient or the like.

The terms “amorphous”, “crystalline”, “solid dispersion”, “amorphous solid dispersion”, “crystalline solid dispersion” and “melt” are as defined above. In addition, the specific crystalline forms of the compound of formula (I) (i.e. forms I, II and III of Ledipasvir) and the specific crystalline forms of the compound of formula (II) (i.e. forms I, VI and VII of Sofosbuvir) are as defined above.

1.3.1 the Compound of Formula (I) is Amorphous

Preferably, the compound of formula (I) is amorphous and the compound of formula (II) is a solid dispersion.

Preferably, the compound of formula (I) is amorphous and the compound of formula (II) is an amorphous solid dispersion.

Preferably, the compound of formula (I) is amorphous and the compound of formula (II) is a crystalline solid dispersion.

Preferably, the compound of formula (I) is amorphous and the compound of formula (II) is a crystalline solid dispersion comprising the crystalline Form I, the crystalline Form VI or the crystalline Form VII, or mixtures thereof.

Preferably, the compound of formula (I) is amorphous and the compound of formula (II) is a crystalline solid dispersion comprising the crystalline Form VII.

Preferably, the compound of formula (I) is amorphous and the compound of formula (II) is a melt.

Preferably, the compound of formula (I) is amorphous and the compound of formula (II) is a melt comprising the compound of formula (II) and at least one pharmaceutically acceptable matrix.

1.3.2 the Compound of Formula (I) is Crystalline

Preferably, the compound of formula (I) is crystalline and the compound of formula (II) is a solid dispersion.

Preferably, the compound of formula (I) is crystalline and is the crystalline Form I, the crystalline Form II or the crystalline Form III, or a mixture of two or more of these forms and the compound of formula (II) is a solid dispersion.

Preferably, the compound of formula (I) is crystalline and the compound of formula (II) is an amorphous solid dispersion.

Preferably, the compound of formula (I) is crystalline and is the crystalline Form I, the crystalline Form II or the crystalline Form III, or a mixture of two or more of these forms and the compound of formula (II) is an amorphous solid dispersion.

Preferably, the compound of formula (I) is crystalline and the compound of formula (II) is a crystalline solid dispersion.

Preferably, the compound of formula (I) is crystalline and the compound of formula (II) is a crystalline solid dispersion comprising the crystalline Form I, the crystalline Form VI or the crystalline Form VII of the compound of formula (II), or mixtures thereof.

Preferably, the compound of formula (I) is crystalline and is the crystalline Form I, the crystalline Form II or the crystalline Form III, or a mixture of two or more of these forms and the compound of formula (II) is a crystalline solid dispersion.

Preferably, the compound of formula (I) is crystalline and is the crystalline Form I, the crystalline Form II or the crystalline Form III, or a mixture of two or more of these forms and the compound of formula (II) is a crystalline solid dispersion comprising the crystalline Form I, the crystalline Form VI or the crystalline Form VII of the compound of formula (II), or mixtures thereof.

Preferably, the compound of formula (I) is crystalline and is the crystalline Form I, the crystalline Form II or the crystalline Form III, or mixtures or two or more of these forms and the compound of formula (II) is a crystalline solid dispersion comprising the crystalline Form VII of the compound of formula (II).

Preferably, the compound of formula (I) is crystalline and is the crystalline Form I and the compound of formula (II) is a crystalline solid dispersion comprising the crystalline Form VII of the compound of formula (II).

Preferably, the compound of formula (I) is crystalline and is the crystalline Form II and the compound of formula (II) is a crystalline solid dispersion comprising the crystalline Form VII of the compound of formula (II).

Preferably, the compound of formula (I) is crystalline and is the crystalline Form III and the compound of formula (II) is a crystalline solid dispersion comprising the crystalline Form VII of the compound of formula (II).

Preferably, the compound of formula (I) is crystalline and is the crystalline Form I, the crystalline Form II or the crystalline Form III, or mixtures or two or more of these forms and the compound of formula (II) is a crystalline solid dispersion comprising the crystalline Form I of the compound of formula (II).

Preferably, the compound of formula (I) is crystalline and is the crystalline Form I and the compound of formula (II) is a crystalline solid dispersion comprising the crystalline Form I of the compound of formula (II).

Preferably, the compound of formula (I) is crystalline and is the crystalline Form II and the compound of formula (II) is a crystalline solid dispersion comprising the crystalline Form I of the compound of formula (II).

Preferably, the compound of formula (I) is crystalline and is the crystalline Form III and the compound of formula (II) is a crystalline solid dispersion comprising the crystalline Form I of the compound of formula (II).

Preferably, the compound of formula (I) is crystalline and the compound of formula (II) is a melt.

Preferably, the compound of formula (I) is crystalline and is the crystalline Form I, the crystalline Form II or the crystalline Form III, or a mixture of two or more of these forms and the compound of formula (II) is a melt.

Preferably, the compound of formula (I) is crystalline and the compound of formula (II) is a melt further comprising at least one pharmaceutically acceptable matrix.

Preferably, the compound of formula (I) is crystalline and is the crystalline Form I, the crystalline Form II or the crystalline Form III, or a mixture of two or more of these forms and the compound of formula (II) is a melt further comprising at least one pharmaceutically acceptable matrix.

1.3.3 the Compound of Formula (II) is Amorphous

Preferably, the compound of formula (I) is a solid dispersion and the compound of formula (II) is amorphous.

Preferably, the compound of formula (I) is an amorphous solid dispersion and the compound of formula (II) is amorphous.

Preferably, the compound of formula (I) is a crystalline solid dispersion and the compound of formula (II) is amorphous.

Preferably, the compound of formula (I) is a crystalline solid dispersion comprising the crystalline Form I, the crystalline Form II or the crystalline Form III of the compound of formula (I), or a mixture of two or more of these forms and the compound of formula (II) is amorphous.

1.3.4 the Compound of Formula (II) is Crystalline

Preferably, the compound of formula (I) is a solid dispersion and the compound of formula (II) is crystalline.

Preferably, the compound of formula (I) is a solid dispersion and the compound of formula (II) is crystalline and is the crystalline Form I, the crystalline Form VI or the crystalline Form VII, or mixtures of two or more thereof.

Preferably, the compound of formula (I) is an amorphous solid dispersion and the compound of formula (II) is crystalline.

Preferably, the compound of formula (I) is an amorphous solid dispersion and the compound of formula (II) is crystalline and is the crystalline Form I, the crystalline Form VI or the crystalline Form VII, or mixtures of two or more thereof.

Preferably, the compound of formula (I) is a crystalline solid dispersion and the compound of formula (II) is crystalline.

Preferably, the compound of formula (I) is a crystalline solid dispersion and the compound of formula (II) is crystalline and is the crystalline Form I, the crystalline Form VI or the crystalline Form VII, or mixtures thereof.

Preferably, the compound of formula (I) is a crystalline solid dispersion comprising the crystalline Form I, the crystalline Form II or the crystalline Form III of the compound of formula (I), or a mixture of two or more of these forms and the compound of formula (II) is crystalline.

Preferably, the compound of formula (I) is a crystalline solid dispersion comprising the crystalline Form I, the crystalline Form II or the crystalline Form III of the compound of formula (I), or a mixture of two or more of these forms and the compound of formula (II) is crystalline and is the crystalline Form I, the crystalline Form VI or the crystalline Form VII, or mixtures thereof.

Preferably, the compound of formula (I) is a crystalline solid dispersion comprising the crystalline Form I, the crystalline Form II or the crystalline Form III of the compound of formula (I), or a mixture of two or more of these forms and the compound of formula (II) is crystalline and is the crystalline Form VII.

Preferably, the compound of formula (I) is a crystalline solid dispersion comprising the crystalline Form I of the compound of formula (I) and the compound of formula (II) is crystalline and is the crystalline Form VII.

Preferably, the compound of formula (I) is a crystalline solid dispersion comprising the crystalline Form II of the compound of formula (I) and the compound of formula (II) is crystalline and is the crystalline Form VII.

Preferably, the compound of formula (I) is a crystalline solid dispersion comprising the crystalline Form III of the compound of formula (I) and the compound of formula (II) is crystalline and is the crystalline Form VII.

Preferably, the compound of formula (I) is a crystalline solid dispersion comprising the crystalline Form I, the crystalline Form II or the crystalline Form III of the compound of formula (I), or a mixture of two or more of these forms and the compound of formula (II) is crystalline and is the crystalline Form I.

Preferably, the compound of formula (I) is a crystalline solid dispersion comprising the crystalline Form I of the compound of formula (I) and the compound of formula (II) is crystalline and is the crystalline Form I.

Preferably, the compound of formula (I) is a crystalline solid dispersion comprising the crystalline Form II of the compound of formula (I) and the compound of formula (II) is crystalline and is the crystalline Form I.

Preferably, the compound of formula (I) is a crystalline solid dispersion comprising the crystalline Form III of the compound of formula (I) and the compound of formula (II) is crystalline and is the crystalline Form I.

1.4 the Preparation of Crystalline Form VII of the Compound of Formula (II)

The present invention also relates to the pharmaceutical composition of the invention wherein the compound of formula (II) is crystalline and is the crystalline Form VII, or wherein the compound of formula (II) is a crystalline solid dispersion comprising crystalline Form VII of the compound of formula (II), said crystalline Form VII of the compound of formula (II) being obtainable or obtained for example by a process comprising

(i) providing the compound of formula (II) in crystalline form, pseudo-crystalline form, amorphous form, or as a mixture of two or more of these forms;

(ii) preparing seed crystals comprising the crystalline Form VII of the compound of formula (II) by a method comprising for example
(ii.1) providing the compound of formula (II) in crystalline form, pseudo-crystalline form, amorphous form, or as a mixture of two or more of these forms;
(ii.2) providing seed crystals of crystalline form VI of the compound of formula (II), having an X-ray powder diffraction pattern with reflections at 2-theta values of (6.1±0.2)°, (8.2±0.2)°, (10.4±0.2)°, (12.7±0.2)°, (20.8±0.2)°, when measured at a temperature in the range of from 15 to 25° C. with Cu-Kalpha1,2 radiation having a wavelength of 0.15419 nm;
(ii.3) preparing a solution of the compound of formula (II) provided in (ii.1) in a C2-C10 alcohol or in a mixture of two or more thereof;
(ii.4) subjecting the solution provided in (ii.3) to crystallization conditions, comprising seeding the solution with the seed crystals provided in (ii.2), wherein during crystallization, the solution is not stirred;
(ii.5) separating at least a portion of the crystalline Form VII of the compound of formula (II) from its mother liquor;

(iii) preparing a solution of the compound of formula (II) provided in (i) in a C2-C5 alcohol or in a mixture of two or more thereof, and in one or more antisolvents;

(iv) subjecting the solution provided in (iii) to crystallization conditions, comprising seeding the solution with the seed crystals prepared in (ii), wherein during crystallization, the solution is not stirred, obtaining the crystalline Form VII of the compound of formula (II) in its mother liquor.

(v) preferably separating the crystalline Form VII of the compound of formula (II) from its mother liquor and drying the crystalline Form VII of the compound of formula (II).

1.5 the Preparation of a Solid Dispersion of the Compound of Formula (II)

The present invention also relates to a pharmaceutical composition wherein the compound of formula (II) is a solid dispersion obtainable or obtained by a process comprising embedding the compound of formula (II) in a matrix consisting of at least one pharmaceutically acceptable matrix compound, starting from a solution of the compound of formula (II) in at least one solvent, wherein the weight ratio of the compound of formula (II) relative to the at least one matrix compound is at least 5.5:4.5, preferably in the range of from 5.5:4.5 to 9:1, more preferably in the range of from 6:4 to 8.5:1.5, more preferably in the range of from 7:3 to 8.5:1.5.

1.6 the Solid Dispersions and Melts

The mixtures, solid dispersions and melts of the invention described above can be further described as follows:

The present invention relates to the pharmaceutical composition of the invention wherein the solid dispersion further comprises at least one pharmaceutically acceptable matrix comprising or consisting of a pharmaceutically acceptable polymer.

The present invention relates to the pharmaceutical composition of the invention wherein the melt further comprises at least one pharmaceutically acceptable matrix comprising or consisting of a pharmaceutically acceptable polymer.

Preferably, the polymer is a water soluble polymer.

Preferably, the polymer is a non-ionic polymer.

Preferably, the polymer is selected from the group consisting of hypromellose, copovidone and povidone.

Preferably, the polymer is copovidone.

Preferably, the polymer is an ionic polymer.

Preferably, the ionic polymer is selected from the group consisting of hydroxypropylmethylcellulose acetate-succinate, hydroxypropylmethylcellulose phthalate and cellulose acetate phthalate.

Preferably, the polymer has a melting point which is lower than the melting point of the compound of formula (II) as defined above in 1.2.2.

Preferably, the polymer has a melting point which is lower than the melting point of the crystalline Form I of the compound of formula (II) as defined above in 1.2.2.

Preferably, the polymer has a melting point which is lower than the melting point of the crystalline Form VII of the compound of formula (II) as defined above in 1.2.2.

1.7 Pharmaceutical Compositions—Amounts

The present invention also relates to the pharmaceutical composition of the invention wherein the weight ratio of the compound of formula (I) to the compound of formula (II) is in the range of from 1:5 to 1:3.5, preferably in the range of from 1:4.5 to 1:4.3, more preferably wherein the weight ratio is 1:4.4.

Preferably, the present invention relates to a pharmaceutical composition comprising the compound of formula (I) in an amount of from 5 to 15 weight %, preferably of from 7 to 12 weight %, more preferably of 9 weight %, based on the total weight of the tablet.

Preferably, the present invention relates to a pharmaceutical composition comprising the compound of formula (II) in an amount of from 30 to 50 weight %, preferably of from 35 to 45 weight %, more preferably of 40 weight %, based on the total weight of the tablet.

Preferably, the present invention relates to a pharmaceutical composition comprising the compound of formula (I) in an amount of 90 mg.

Preferably, the present invention relates to a pharmaceutical composition comprising the compound of formula (II) in an amount of 400 mg.

Preferably, the present invention relates to a pharmaceutical composition comprising the compound of formula (I) in an amount of 90 mg and the compound of formula (II) in an amount of 400 mg.

1.8 Pharmaceutical Compositions—Further Components

The present invention also relates to the pharmaceutical composition of the invention further comprising at least one HCV agent other than the compound of formula (I) or the compound of formula (II).

Preferably, the at least one HCV agent other than the compound of formula (I) or the compound of formula (II) is Telaprevir, Daclatasvir, Simeprevir, Boceprevir, ABT-450, Dasabuvir, Ombitasvir, Velpatasvir or any mixture of two or more thereof, optionally in combination with suitable agents such as Ribavirin or PEG-Interferon.

In addition to the compound of formula (I) or the compound of formula (II), the pharmaceutical compositions of the present invention may further comprise at least one pharmaceutically acceptable excipient.

The term “pharmaceutically acceptable excipient” as used in this context of the present invention relates to a compound that is generally safe, non-toxic and neither biologically nor otherwise undesirable, and includes excipients that are acceptable for human pharmaceutical use.

With regard to the at least one excipient, no specific restrictions exist provided that a pharmaceutical composition with the desired properties is obtained. Conceivable excipients include diluents, disintegrants, glidants, lubricants, coloring agents, taste-masking agents, coating agents, and the like.

Thus, the present invention relates to a pharmaceutical composition wherein the at least one pharmaceutically acceptable excipient comprises at least one diluent, or at least one disintegrant, or at least one glidant, or at least one lubricant, or a combination of at least one diluent and at least one disintegrant, or a combination of at least one diluent and at least one glidant, or a combination of at least one disintegrant and at least one lubricant, or a combination of at least one diluent and at least one disintegrant and at least one glidant, or a combination of at least one diluent and at least one disintegrant and at least one lubricant, or a combination of at least one disintegrant and at least one glidant and at least one lubricant, or a combination of at least one diluent and at least one disintegrant and at least one glidant and at least one lubricant, wherein the at least one pharmaceutically acceptable excipient preferably comprises a combination of at least one diluent and at least one disintegrant and at least one glidant and at least one lubricant.

With regard to the at least one diluent, no specific restrictions exist provided that a pharmaceutical composition with the desired properties is obtained.

Preferably, the present invention relates to a pharmaceutical composition wherein the at least one diluent comprises, preferably is, at least one of calcium carbonate, dicalcium phosphate, dry starch, calcium sulfate, cellulose, compressible sugars, confectioner's sugar, dextrates, dextrin, dextrose, dibasic calcium phosphate dihydrate, glyceryl palmitostearate, hydrogenated vegetable oil type I, inositol, kaolin, lactose, magnesium carbonate, magnesium oxide, maltodextrin, mannitol, microcrystalline cellulose, polymethacrylates, potassium chloride, powdered cellulose, powdered sugar, pregelatinized starch, sodium chloride, sorbitol, starch, sucrose, sugar spheres, talc, tribasic calcium phosphate, preferably at least one of dicalcium phosphate, cellulose, compressible sugars, dibasic calcium phosphate dehydrate, lactose, mannitol, microcrystalline cellulose, starch, tribasic calcium phosphate, more preferably at least one of mannitol, microcrystalline cellulose.

More preferably, the present invention relates to a pharmaceutical composition wherein the at least one diluent comprises, preferably is, a combination of mannitol and microcrystalline cellulose.

With regard to the at least one disintegrant, no specific restrictions exist provided that a pharmaceutical composition with the desired properties is obtained.

Preferably, the present invention relates to a pharmaceutical composition wherein the at least disintegrant comprises, preferably is, at least one of agar, alginic acid, bentonite, carboxymethylcellulose calcium, carboxymethylcellulose sodium, carboxymethylcellulose, cellulose, a cation exchange resin, cellulose, gums, citrus pulp, colloidal silicon dioxide, corn starch, croscarmellose sodium, crospovidone, guar gum, hydrous aluminum silicate, an ion exchange resin (e.g., polyacrin potassium), magnesium aluminum silicate, methyl cellulose, microcrystalline cellulose, modified cellulose gum, modified corn starch, montmorillonite clay, natural sponge, polyacrilin potassium, potato starch, powdered cellulose, povidone, pregelatinized starch, sodium alginate, sodium bicarbonate in admixture with an acidulant such as tartaric acid or citric acid, sodium starch glycolate, starch, silicates, preferably at least one of croscarmellose sodium, crospovidone, microcrystalline cellulose, modified corn starch, povidone, pregelatinized starch, sodium starch glycolate.

More preferably, the present invention relates to a pharmaceutical composition wherein the at least one disintegrant comprises, preferably is, croscarmellose sodium.

With regard to the at least one glidant, no specific restrictions exist provided that a pharmaceutical composition with the desired properties is obtained.

Preferably, the present invention relates to a pharmaceutical composition wherein the at least one glidant comprises, preferably is, is at least one of colloidal silicon dioxide, talc, starch, starch derivatives.

More preferably, the present invention relates to a pharmaceutical composition wherein the at least one glidant comprises, preferably is, colloidal silicon dioxide.

With regard to the at least one lubricant, no specific restrictions exist provided that a pharmaceutical composition with the desired properties is obtained.

Preferably, the present invention relates to a pharmaceutical composition wherein the at least one lubricant comprises, preferably is, at least one of calcium stearate, glyceryl monostearate, glyceryl palmitostearate, hydrogenated castor oil, hydrogenated vegetable oil, light mineral oil, magnesium stearate, mineral oil, polyethylene glycol, sodium benzoate, sodium lauryl sulfate, sodium stearyl fumarate, stearic acid, talc, zinc stearate, preferably at least one of calcium stearate, magnesium stearate, polyethylene glycol, sodium stearyl fumarate, stearic acid, talc.

More preferably, the present invention relates to a pharmaceutical composition wherein the at least one lubricant comprises, preferably is, magnesium stearate.

The pharmaceutical compositions described in the present invention can also be in the form of a tablet.

Preferably, they can be in the form of a tablet for oral administration. Thus, the pharmaceutical compositions of the present invention comprising at least one pharmaceutically acceptable excipient may further comprise at least one coating agent.

With regard to the at least one coating agent, no specific restrictions exist provided that a pharmaceutical composition with the desired properties is obtained.

Preferably, the present invention relates to a pharmaceutical composition wherein the at least one pharmaceutically acceptable excipient further comprises at least one coating agent. The coating agent can be formed from an aqueous film coat composition, wherein the aqueous film coat composition may comprise a film-forming polymer, water and/or an alcohol as a vehicle, and optionally one or more adjuvants such as are known in the film-coating art.

More preferably, the present invention relates to a pharmaceutical composition wherein the at least one coating agent comprises, preferably is, at least one of hydroxypropylmethylcellulose, hydroxypropylcellulose, methylcellulose, ethylcellulose, hydroxyethylcellulose, cellulose acetate phthalate, sodium ethyl cellulose sulfate, carboxymethyl cellulose, polyvinylpyrolidone, zein, an acrylic polymer including methacrylic acid or methacrylic acid ester copolymers including methacrylic acid or methylmethacrylate copolymers, a polyvinyl alcohol.

More preferably, the present invention relates to a pharmaceutical composition wherein the at least one coating agent comprises, preferably is, a polyvinyl alcohol.

The coating agent may further comprise a taste-masking agent. In this case, the coating agent may be formed from an aqueous film coat composition, wherein the aqueous film coat com-position may comprise a film-forming polymer, water and/or an alcohol as a vehicle, and optionally one or more adjuvants such as are known in the film-coating art.

Thus preferably, the present invention relates to a pharmaceutical composition wherein the at least one coating agent comprises at least one taste-masking agent.

More preferably, the present invention relates to a pharmaceutical composition wherein the at least one coating agent comprises, preferably is, a combination of a polyvinyl alcohol and at least one taste-masking agent.

2. Process for the Preparation of Pharmaceutical Compositions Comprising the Compound of Formula (I) and the Compound of Formula (II)

The present invention relates to processes for the preparation of the pharmaceutical compositions as described above, preferably of solid pharmaceutical compositions, preferably of solid pharmaceutical compositions in the form of a tablet, comprising the steps of:

(i) providing a compound of formula (I) or a pharmaceutically acceptable solvate or salt thereof

(ii) providing a compound of formula (II) or a pharmaceutically acceptable salt thereof

(iii) mixing the compound of formula (I) or a pharmaceutically acceptable solvate or salt thereof and the compound of formula (II) or a pharmaceutically acceptable salt thereof

(iv) optionally blending the mixture provided in (iii) with at least one pharmaceutically acceptable excipient and

(v) optionally preparing a tablet based on the blend obtained in (iv)

wherein the compound of formula (I) and the compound of formula (II) can be in crystalline or amorphous form, and wherein when the compound of formula (I) is amorphous and the compound of formula (II) is crystalline the compound of formula (II) is the crystalline Form VII.

The terms “amorphous” and “crystalline” are as defined above. In addition, the specific crystalline forms of the compound of formula (I) (i.e. Forms I, II and III of Ledipasvir) and the specific crystalline forms of the compound of formula (II) (i.e. Forms I, VI and VII of Sofosbuvir) are as defined above.

2.1 the Compound of Formula (I) and/or the Compound of Formula (II) is/are Amorphous and/or Crystalline

The present invention also relates to a process wherein the compound of formula (I) is amorphous.

The present invention also relates to a process wherein the compound of formula (I) is crystalline.

Preferably, the compound of formula (I) is crystalline and is the crystalline Form I, the crystalline Form II or the crystalline Form III, or a mixture of two or more of these forms.

The present invention also relates to a process wherein the compound of formula (II) is amorphous.

The present invention also relates to a process wherein the compound of formula (II) is crystalline.

Preferably, the compound of formula (II) is crystalline and is the crystalline Form I, the crystalline Form VI or the crystalline Form VII, or a mixture thereof.

The present invention also relates to a process wherein the compound of formula (I) is amorphous and the compound of formula (II) is amorphous.

The present invention also relates to a process wherein the compound of formula (I) is crystalline and the compound of formula (II) is amorphous.

Preferably, the compound of formula (I) is crystalline and is the crystalline Form I, the crystalline Form II or the crystalline Form III, or a mixture of two or more of these forms and the compound of formula (II) is amorphous.

Preferably, the compound of formula (I) is crystalline and comprises a mixture of two or more crystalline forms and the compound of formula (II) is amorphous.

Preferably, the compound of formula (I) is crystalline and comprises a mixture of two or more crystalline forms selected from the crystalline Form I, the crystalline Form II or the crystalline Form III, or mixtures thereof and the compound of formula (II) is amorphous.

Preferably, the compound of formula (I) is crystalline and is the crystalline Form I and the compound of formula (II) is amorphous.

Preferably, the compound of formula (I) is crystalline and is the crystalline Form II and the compound of formula (II) is amorphous.

Preferably, the compound of formula (I) is crystalline and is the crystalline Form III and the compound of formula (II) is amorphous.

The present invention also relates to a process wherein the compound of formula (I) is crystalline and the compound of formula (II) is crystalline.

Preferably, the compound of formula (I) is crystalline and the compound of formula (II) is crystalline and is the crystalline Form I, the crystalline Form VI or the crystalline Form VII, or a mixture thereof.

Preferably, the compound of formula (I) is crystalline and is the crystalline Form I, the crystalline Form II or the crystalline Form III or a mixture of two or more of these forms and the compound of formula (II) is crystalline.

Preferably, the compound of formula (I) is crystalline and is the crystalline Form I, the crystalline Form II or the crystalline Form III or a mixture of two or more of these forms and the compound of formula (II) is crystalline and is the crystalline Form I, the crystalline Form VI or the crystalline Form VII or a mixture thereof.

Preferably, the compound of formula (I) is crystalline and comprises a mixture of two or more crystalline forms and the compound of formula (II) is crystalline.

Preferably, the compound of formula (I) is crystalline and comprises a mixture of two or more crystalline forms selected from the crystalline Form I, the crystalline Form II and the crystalline Form III and the compound of formula (II) is crystalline.

Preferably, the compound of formula (I) is crystalline and comprises a mixture of two or more crystalline forms and the compound of formula (II) is crystalline and is the crystalline Form I, the crystalline Form VI or the crystalline Form VII.

Preferably, the compound of formula (I) is crystalline and comprises a mixture of two or more crystalline forms selected from the crystalline Form I, the crystalline Form II and the crystalline Form III and the compound of formula (II) is crystalline and is the crystalline Form I, the crystalline Form VI or the crystalline Form VII.

Preferably, the compound of formula (I) is crystalline and comprises a mixture of two or more crystalline forms selected from the crystalline Form I, the crystalline Form II and the crystalline Form III and the compound of formula (II) is crystalline and is the crystalline Form VII.

Preferably, the compound of formula (I) is crystalline and is the crystalline Form I and wherein the compound of formula (II) is crystalline and is the crystalline Form VII.

Preferably, the compound of formula (I) is crystalline and is the crystalline Form II and wherein the compound of formula (II) is crystalline and is the crystalline Form VII.

Preferably, the compound of formula (I) is crystalline and is the crystalline Form III and wherein the compound of formula (II) is crystalline and is the crystalline Form VII.

The present invention also relates to a process wherein the compound of formula (I) is amorphous and the compound of formula (II) is crystalline and is the crystalline Form I.

Preferably, the compound of formula (I) is crystalline and comprises a mixture of two or more crystalline forms selected from the crystalline Form I, the crystalline Form II and the crystalline Form III and the compound of formula (II) is crystalline and is the crystalline Form I.

Preferably, the compound of formula (I) is crystalline and is the crystalline Form I and wherein the compound of formula (II) is crystalline and is the crystalline Form I.

Preferably, the compound of formula (I) is crystalline and is the crystalline Form II and wherein the compound of formula (II) is crystalline and is the crystalline Form I.

Preferably, the compound of formula (I) is crystalline and is the crystalline Form III and wherein the compound of formula (II) is crystalline and is the crystalline Form I.

The present invention also relates to a process wherein the compound of formula (I) is amorphous and the compound of formula (II) is crystalline and is the crystalline Form I.

2.2 the Compound of Formula (I) and/or the Compound of Formula (II) is/are a Solid Dispersion and/or a Melt

For the purposes of this invention, the compound of formula (I) and the compound of formula (II) can also be provided as a solid dispersion, specifically as an amorphous solid dispersion or as a crystalline solid dispersion. The terms “solid dispersion”, “amorphous solid dispersion” and “crystalline solid dispersion” are as defined above. The specific crystalline forms of the compound of formula (I) (i.e. Forms I, II and Ill of Ledipasvir) and the specific crystalline forms of the compound of formula (II) (i.e. Forms I, VI and VII of Sofosbuvir) are as defined above.

Thus, the present invention relates to a process wherein step (i) comprises providing a solid dispersion of the compound of formula (I) or of the compound of formula (II).

Preferably, the present invention relates to a process wherein step (i) comprises providing an amorphous solid dispersion of the compound of formula (I) or of the compound of formula (II).

Preferably, the present invention relates to a process wherein step (i) comprises providing an amorphous solid dispersion of the compound of formula (I) and of the compound of formula (II).

Preferably, step (i) comprises providing a crystalline solid dispersion of the compound of formula (I) or of the compound of formula (II).

Preferably, step (i) comprises providing a crystalline solid dispersion of the compound of formula (I) and of the compound of formula (II).

2.2.1 the Compound of Formula (I) and/or the Compound of Formula (II) is/are an Amorphous Solid Dispersion

Regarding the provision of an amorphous solid dispersion of the compound of formula (I) or of the compound of formula (II), the present invention relates to a process wherein step (i) comprises the steps of

(1.1) providing a compound of formula (I) or a compound of formula (II) and a pharmaceutically acceptable matrix,

(1.2) dissolving the compound of formula (I) or the compound of formula (II) and the pharmaceutically acceptable matrix in at least one suitable solvent,

(1.3) removing the at least one suitable solvent,

(1.4) optionally milling and drying the solid resulting from (1.3),

wherein in step (1.1) the compound of formula (I) or the compound of formula (II) are in amorphous or crystalline form and wherein in step (1.2) the compound of formula (I) or the compound of formula (II) and the pharmaceutically acceptable matrix are completely dissolved.

In the context of the present invention, the term “completely dissolved” is to be understood that at least 85%, preferably at least 90%, preferably at least 95%, preferably at least 99%, preferably at least 99.9%, preferably at least 99.99%, preferably at least 99.999% of each of the compound of formula (I) or of the compound of formula (II) and of the pharmaceutically acceptable matrix are dissolved in the at least one suitable solvent.

Regarding the nature of the compound of formula (I) and of the compound of formula (II), these compounds can be either amorphous or crystalline. The terms “amorphous” and “crystalline” are as defined above. The specific crystalline forms of the compound of formula (I) (i.e. Forms I, II and III of Ledipasvir) and the specific crystalline forms of the compound of formula (II) (i.e. Forms I, VI and VII of Sofosbuvir) are as defined above. Thus, the compound of formula (I) can be provided in amorphous or in crystalline form or mixtures thereof. If crystalline, it can be provided as the crystalline Form I, II, III or mixtures thereof. The compound of formula (II) can be provided in crystalline or in amorphous form or mixtures thereof. If crystalline, it can be provided as the crystalline Form I, VI or VII or mixtures thereof.

Regarding the weight ratio of the compound of formula (I) or of the compound of formula (II) to the at least one suitable solvent, no specific restrictions exist provided that an amorphous solid dispersion of the compound of formula (I) or of the compound of formula (II) is obtained.

Preferably, in step (1.2) the weight ratio of the compound of formula (I) or of the compound of formula (II) to the at least one suitable solvent is of from 1:8 to 1:15, preferably of from 1:8 to 1:12.

Regarding the at least one suitable solvent, no specific restrictions exist provided that an amorphous solid dispersion of the compound of formula (I) or of the compound of formula (II) is obtained.

Preferably, in step (1.2) the solvent is a polar solvent.

Preferably, in step (1.2) the solvent is a polar protic solvent.

Preferably, in step (1.2) the solvent is selected from the list consisting of acetone, a C1 alcohol, a C2 alcohol, a C3 alcohol, or a mixture of two or more thereof. Preferably, in (1.2) the solvent is selected from a C2 alcohol and acetone. Preferably, the present invention relates to a process wherein in (1.2) the solvent is a C2 alcohol. Preferably, the present invention relates to a process wherein in (1.2) the solvent is acetone.

Regarding the temperature in (1.3), no specific restrictions exist provided that an amorphous solid dispersion of the compound of formula (I) or of the compound of formula (II) is obtained.

Preferably, in step (1.3) the temperature is in the range of from 20 to 150 degrees Celsius, preferably in the range of from 20 to 120 degrees Celsius, preferably in the range of from 20 to 100 degrees Celsius, preferably in the range of from 20 to 80 degrees Celsius, preferably in the range of from 20 to 60 degrees Celsius, preferably in the range of from 25 to 55 degrees Celsius, preferably in the range of from 25 to 50 degrees Celsius, preferably in the range of from 20 to 40 degrees Celsius.

Regarding the method for removing the at least one suitable solvent in (1.3), no specific restrictions exist provided that an amorphous solid dispersion of the compound of formula (I) or of the compound of formula (II) is obtained.

Preferably, in step (1.3) removing the solvent comprises spray drying, lyophilization or rotary evaporation.

2.2.2 the Compound of Formula (I) and/or the Compound of Formula (II) is/are a Crystalline Solid Dispersion

Regarding the provision of a crystalline solid dispersion of the compound of formula (I) or of the compound of formula (II), the present invention relates to a process (i) comprises the steps of

(2.1) providing a suitable amount of a pharmaceutically acceptable matrix,

(2.2) dissolving the pharmaceutically acceptable matrix of step (2.1) in at least one suitable solvent,

(2.3) adding a suitable amount of the compound of formula (I) or of the compound of formula (II),

(2.4) removing the at least one suitable solvent,

(2.5) optionally milling and drying the solid resulting from (2.4),

wherein in (2.2) the amount of the at least one suitable solvent is chosen so that after step (2.3) substantially all of the compound of formula (I) or of the compound of formula (II) is undissolved.

In the context of the present invention, the term “undissolved” is to be understood that at least 85%, preferably at least 90%, preferably at least 95%, preferably at least 99%, preferably at least 99.9%, of each of the compound of formula (I) and of the compound of formula (II) are not dissolved in the at least one suitable solvent at the end of step (2.3).

Preferably, in step (2.3) the compound of formula (I) or the compound of formula (II) is crystalline.

Preferably, in step (2.3) the compound of formula (I) is the crystalline Form I, the crystalline Form II or the crystalline Form III or any mixture of two or more thereof.

Preferably, in step (2.3) the compound of formula (I) is crystalline and comprises a mixture of two or more crystalline forms.

Preferably, in step (2.3) the compound of formula (I) is crystalline and comprises a mixture of two or more crystalline forms from the crystalline Form I, the crystalline Form II or the crystalline Form III.

Preferably, in step (2.3) the compound of formula (I) is crystalline and is the crystalline Form I, the crystalline Form II or the crystalline Form III.

Preferably, in step (2.3) the compound of formula (II) is the crystalline Form I, the crystalline Form VI or the crystalline Form VII.

Regarding the at least one suitable solvent, no specific restrictions exist provided that a crystalline solid dispersion of the compound of formula (I) or of the compound of formula (II) is obtained.

Preferably, in step (2.2) the solvent is a polar solvent.

Preferably, in step (2.2) the solvent is a polar protic solvent.

Preferably, the present invention relates to a process wherein in (1.2) the solvent is selected from water, acetone, a C1 alcohol, a C2 alcohol, a C3 alcohol, or a mixture of two or more thereof. Preferably, in step (1.2) the solvent is selected from water, a C2 alcohol and acetone or any mixture of two or more thereof.

Preferably, in step (1.2) the solvent is a C2 alcohol. Preferably, in step (1.2) the solvent is acetone.

Preferably, in step (1.2) the solvent is water. Preferably, in step (1.2) the solvent is a mixture of water and Ethanol. Preferably, in step (1.2) the solvent is a mixture of water and acetone.

Regarding the temperature in (2.4), no specific restrictions exist provided that a crystalline solid dispersion of the compound of formula (I) or of the compound of formula (II) is obtained.

Preferably, in step (2.4) the temperature is in the range of from 20 to 150 degrees Celsius, preferably in the range of from 20 to 120 degrees Celsius, preferably in the range of from 20 to 100 degrees Celsius, preferably in the range of from 20 to 80 degrees Celsius, preferably in the range of from 20 to 60 degrees Celsius, preferably in the range of from 25 to 55 degrees Celsius, preferably in the range of from 25 to 50 degrees Celsius, preferably in the range of from 20 to 40 degrees Celsius.

Regarding the method for removing the at least one suitable solvent in (2.4), no specific restrictions exist provided that a crystalline solid dispersion of the compound of formula (I) or of the compound of formula (II) is obtained.

Preferably, in step (2.4) removing the solvent comprises spray drying, lyophilization or rotary evaporation.

2.2.3 the Compound of Formula (II) is a Melt

The present invention also relates to a process wherein (ii) comprises providing a melt of the compound of formula (II). The term “melt” is as defined above. In the context of the present invention, the term “melting conditions” as used herein refers to conditions comprising subjecting the compound of formula (II) or any mixture comprising the compound of formula (II) to a temperature sufficient to completely melt said compound of formula (II). Preferably, the term “melting conditions” as used herein refers to conditions comprising subjecting the compound of formula (II) or any mixture comprising the compound of formula (II) to a temperature sufficient to completely melt only said compound of formula (II). Therefore, it is to be understood in the context of the present invention that at least 80%, preferably at least 85%, preferably at least 90%, preferably at least 95%, preferably at least 99%, preferably at least 99.9%, preferably at least 99.99%, preferably at least 99.999% of the compound of formula (II) is molten at the end of the corresponding process step.

Preferably, step (ii) comprises providing an amorphous melt of the compound of formula (II).

Preferably, step (ii) comprises

(3.1) providing a suitable amount of the compound of formula (II) or a mixture comprising a suitable amount of the compound of formula (II) and a pharmaceutically acceptable matrix,

(3.2) subjecting the compound provided in (3.1) to melting conditions,

(3.3) cooling the mixture obtained in (3.2) until it completely solidifies,

(3.4) optionally milling the mixture obtained in (3.3),

(3.5) optionally sieving the mixture obtained in (3.3) or (3.4).

Preferably, in step (3.1) the compound of formula (II) is amorphous.

Preferably, in step (3.1) the compound of formula (II) is crystalline.

Preferably, in step (3.1) the compound of formula (II) is crystalline and is the crystalline Form I, the crystalline Form VI or the crystalline Form VII. Preferably, the compound of formula (II) is provided in amorphous form or as the crystalline Form I.

Preferably, in step (3.1) providing a suitable amount of the compound of formula (II) comprises providing a mixture comprising a suitable amount of the compound of formula (II) and at least one pharmaceutically acceptable matrix, wherein the matrix is as defined below in 2.2.5.

The present invention also relates to a process wherein step (iii) further comprises

(4.1) subjecting the mixture provided in (iii) to melting conditions,

(4.2) cooling the mixture obtained in (4.2) until it completely solidifies,

(4.3) optionally milling the mixture obtained in (4.3),

(4.4) optionally sieving the mixture obtained in (4.3) or (4.4),

Regarding the nature of the compound of formula (I) and of the compound of formula (II), these compounds can be either amorphous or crystalline. The terms “amorphous” and “crystalline” are as defined above. The specific crystalline forms of the compound of formula (I) (i.e. Forms I, II and III of Ledipasvir) and the specific crystalline forms of the compound of formula (II) (i.e. Forms I, VI and VII of Sofosbuvir) are as defined above. Thus, the compound of formula (I) can be provided in amorphous or in crystalline form or mixtures thereof. If crystalline, it can be provided as the crystalline Form I, II, III or mixtures thereof. The compound of formula (II) can be provided in crystalline or in amorphous form or mixtures thereof. If crystalline, it can be provided as the crystalline Form I, VI or VII or mixtures thereof.

Preferably, the compound of formula (II) is provided in amorphous form or as the crystalline Form I.

The present invention also relates to a process wherein step (i) comprises

(5.1) providing a mixture comprising a suitable amount of the compound of formula (I) and wherein step (iii) further comprises

(5.2) subjecting the mixture obtained from (i) and (ii) to melting conditions,

(5.3) cooling the mixture obtained in (5.2) until it completely solidifies,

(5.4) optionally milling the mixture obtained in (5.3),

(5.5) optionally sieving the mixture obtained in (5.3) or (5.4).

Regarding the nature of the compound of formula (I) and of the compound of formula (II), these compounds can be either amorphous or crystalline. The terms “amorphous” and “crystalline” are as defined above. The specific crystalline forms of the compound of formula (I) (i.e. Forms I, II and III of Ledipasvir) and the specific crystalline forms of the compound of formula (II) (i.e. Forms I, VI and VII of Sofosbuvir) are as defined above. Thus, the compound of formula (I) can be provided in amorphous or in crystalline form or mixtures thereof. If crystalline, it can be provided as the crystalline Form I, II, III or mixtures thereof. The compound of formula (II) can be provided in crystalline or in amorphous form or mixtures thereof. If crystalline, it can be provided as the crystalline Form I, VI or VII or mixtures thereof.

Preferably, the compound of formula (II) is provided in amorphous form or as the crystalline Form I.

The present invention also relates to a process wherein (ii) comprises

(6.1) providing a mixture comprising a suitable amount of the compound of formula (II) and wherein (iii) comprises

(6.2) subjecting the mixture obtained from (i) and (ii) to melting conditions,

(6.3) cooling the mixture obtained in (6.2) until it completely solidifies,

(6.4) optionally milling the mixture obtained in (6.3),

(6.5) optionally sieving the mixture obtained in (6.3) or (6.4).

Regarding the nature of the compound of formula (I) and of the compound of formula (II), these compounds can be either amorphous or crystalline. The terms “amorphous” and “crystalline” are as defined above. The specific crystalline forms of the compound of formula (I) (i.e. Forms I, II and III of Ledipasvir) and the specific crystalline forms of the compound of formula (II) (i.e. Forms I, VI and VII of Sofosbuvir) are as defined above. Thus, the compound of formula (I) can be provided in amorphous or in crystalline form or mixtures thereof. If crystalline, it can be provided as the crystalline Form I, II, III or mixtures thereof. The compound of formula (II) can be provided in crystalline or in amorphous form or mixtures thereof. If crystalline, it can be provided as the crystalline Form I, VI or VII or mixtures thereof.

Preferably, the compound of formula (II) is provided in amorphous form or as the crystalline Form I.

The present invention also relates to a process wherein (i) comprises

(7.1) providing a mixture comprising a suitable amount of the compound of formula (I) and wherein (ii) comprises

(7.2) providing a mixture comprising a suitable amount of the compound of formula (II) and wherein (iii) comprises

(7.3) subjecting the mixture obtained from (i) and (ii) to melting conditions

(7.4) cooling the mixture obtained in (7.2) until it completely solidifies

(7.5) optionally milling the mixture obtained in (7.4)

(7.6) optionally sieving the mixture obtained in (7.4) or (7.5).

Regarding the nature of the compound of formula (I) and of the compound of formula (II), these compounds can be either amorphous or crystalline. The terms “amorphous” and “crystalline” are as defined above. The specific crystalline forms of the compound of formula (I) (i.e. Forms I, II and III of Ledipasvir) and the specific crystalline forms of the compound of formula (II) (i.e. Forms I, VI and VII of Sofosbuvir) are as defined above. Thus, the compound of formula (I) can be provided in amorphous or in crystalline form or mixtures thereof. If crystalline, it can be provided as the crystalline Form I, II, III or mixtures thereof. The compound of formula (II) can be provided in crystalline or in amorphous form or mixtures thereof. If crystalline, it can be provided as the crystalline Form I, VI or VII or mixtures thereof. Preferably, the compound of formula (II) is provided in amorphous form or as the crystalline Form I.

Preferably, in step (5.1) or in step (7.1) the mixture comprising a suitable amount of the compound of formula (I) is a solid dispersion comprising the compound of formula (I) and a pharmaceutically acceptable matrix.

Preferably, in step (5.1) or in step (7.1) the mixture comprising a suitable amount of the compound of formula (I) is a crystalline solid dispersion comprising the compound of formula (I) and a pharmaceutically acceptable matrix.

Preferably, in step (5.1) or in step (7.1) the mixture comprising a suitable amount of the compound of formula (I) is an amorphous solid dispersion comprising the compound of formula (I) and a pharmaceutically acceptable matrix.

Preferably, the solid dispersion comprising the compound of formula (I) and a pharmaceutically acceptable matrix is prepared according to any of the processes described above.

Preferably, in step (6.1) or in step (7.2) the mixture comprising a suitable amount of the compound of formula (II) is a solid dispersion comprising the compound of formula (II) and a pharmaceutically acceptable matrix.

Preferably, in step (6.1) or in step (7.2) the mixture comprising a suitable amount of the compound of formula (II) is a crystalline solid dispersion comprising the compound of formula (II) and a pharmaceutically acceptable matrix.

Preferably, in step (6.1) or in step (7.2) the mixture comprising a suitable amount of the compound of formula (II) is an amorphous solid dispersion comprising the compound of formula (II) and a pharmaceutically acceptable matrix.

Preferably, the solid dispersion comprising the compound of formula (II) and a pharmaceutically acceptable matrix is prepared according to any of the processes described above.

The present invention also relates to a process wherein steps (i) and (ii) together comprise providing a mixture comprising the compound of formula (I) and the compound of formula (II) and wherein step (iii) comprises

(8.1) subjecting the mixture comprising the compound of formula (I) and the compound of formula (II) to melting conditions

(8.2) cooling the mixture obtained in (8.1) until it completely solidifies

(8.3) optionally milling the mixture obtained in (8.2)

(8.4) optionally sieving the mixture obtained in (8.2) or (8.3).

Regarding the nature of the compound of formula (I) and of the compound of formula (II), these compounds can be either amorphous or crystalline. The terms “amorphous” and “crystalline” are as defined above. The specific crystalline forms of the compound of formula (I) (i.e. Forms I, II and III of Ledipasvir) and the specific crystalline forms of the compound of formula (II) (i.e. Forms I, VI and VII of Sofosbuvir) are as defined above. Thus, the compound of formula (I) can be provided in amorphous or in crystalline form or mixtures thereof. If crystalline, it can be provided as the crystalline Form I, II, III or mixtures thereof. The compound of formula (II) can be provided in crystalline or in amorphous form or mixtures thereof. If crystalline, it can be provided as the crystalline Form I, VI or VII or mixtures thereof. Preferably, the compound of formula (II) is provided in amorphous form or as the crystalline Form I.

Preferably, the mixture comprising the compound of formula (I) and of the compound of formula (II) is a solid dispersion comprising the compound of formula (I), the compound of formula (II) and a pharmaceutically acceptable matrix.

Preferably, the mixture comprising the compound of formula (I) and of the compound of formula (II) is a solid dispersion prepared according to any of the processes described above.

2.2.4 the Compound of Formula (I) and the Compound of Formula (II) are a Homogeneous Solid Dispersion

The present invention also relates to a process wherein (iii) comprises

(9.1) adding a suitable amount of a pharmaceutically acceptable matrix to the compound of formula (I) and to the compound of formula (II) provided in (i) and (ii),

(9.2) adding a suitable amount of at least one suitable solvent

(9.3) removing the at least one solvent

(9.4) optionally milling and drying the solid resulting from (9.3)

Regarding the nature of the compound of formula (I) and of the compound of formula (II), these compounds can be either amorphous or crystalline. The terms “amorphous” and “crystalline” are as defined above. The specific crystalline forms of the compound of formula (I) (i.e. Forms I, II and III of Ledipasvir) and the specific crystalline forms of the compound of formula (II) (i.e. Forms I, VI and VII of Sofosbuvir) are as defined above. Thus, the compound of formula (I) can be provided in amorphous or in crystalline form or mixtures thereof. If crystalline, it can be provided as the crystalline Form I, II, III or mixtures thereof. The compound of formula (II) can be provided in crystalline or in amorphous form or mixtures thereof. If crystalline, it can be provided as the crystalline Form I, VI or VII or mixtures thereof.

Preferably, the compound of formula (II) is provided in amorphous form or as the crystalline Form I.

Preferably, after step (9.2) the compound of formula (I) and the compound of formula (II) and the pharmaceutically acceptable matrix are completely dissolved.

Preferably, after step (9.2) the compound of formula (I) and the pharmaceutically acceptable matrix are completely dissolved.

Preferably, after step (9.2) the compound of formula (II) and the pharmaceutically acceptable matrix are completely dissolved.

Preferably, after step (9.2) substantially all of the compound of formula (I) and the compound of formula (II) remains undissolved.

Regarding the at least one suitable solvent, no specific restrictions exist provided that a homogeneous solid dispersion of the compound of formula (I) and of the compound of formula (II) is obtained.

Preferably, in step (9.2) the solvent is a polar solvent.

Preferably, in step (9.2) the solvent is a polar protic solvent.

Preferably, in step (1.2) the solvent is selected from the list consisting of acetone, a C1 alcohol, a C2 alcohol, a C3 alcohol, or in a mixture of two or more thereof. Preferably, in step (1.2) the solvent is selected from a C2 alcohol and acetone. Preferably, in step (1.2) the solvent is a C2 alcohol. Preferably, in step (1.2) the solvent is acetone.

Regarding the temperature in (9.3), no specific restrictions exist provided that a crystalline solid dispersion of the compound of formula (I) or of the compound of formula (II) is obtained.

Preferably, in step (9.3) the temperature is in the range of from 20 to 150 degrees Celsius, preferably in the range of from 20 to 120 degrees Celsius, preferably in the range of from 20 to 100 degrees Celsius, preferably in the range of from 20 to 80 degrees Celsius, preferably in the range of from 20 to 60 degrees Celsius, preferably in the range of from 25 to 55 degrees Celsius, preferably in the range of from 25 to 50 degrees Celsius, preferably in the range of from 20 to 40 degrees Celsius.

2.2.5 The mixtures, solid dispersions and melts

Preferably, the pharmaceutically acceptable matrix of any of the mixtures, solid dispersions and melts described in this invention comprises or consists of a pharmaceutically acceptable polymer.

Preferably, the pharmaceutically acceptable polymer is a water soluble polymer.

Preferably, the pharmaceutically acceptable polymer is a non-ionic polymer.

Preferably, the pharmaceutically acceptable polymer is selected from the group consisting of hypromellose, copovidone and povidone.

Preferably, the pharmaceutically acceptable polymer is copovidone.

Preferably, the pharmaceutically acceptable polymer is an ionic polymer.

Preferably, the ionic polymer is selected from the group consisting of hydroxypropylmethylcellulose acetate-succinate, hydroxypropylmethylcellulose phthalate and cellulose acetate phthalate.

Preferably, the polymer has a melting point which is lower than the melting point of the compound of formula (II) as defined above in 1.2.2.

Preferably, the polymer has a melting point which is lower than the melting point of the crystalline Form I of the compound of formula (II) as defined above in 1.2.2.

Preferably, the polymer has a melting point which is lower than the melting point of the crystalline Form VII of the compound of formula (II) as defined above in 1.2.2.

Further, the present invention relates to any of the pharmaceutical compositions described above, obtainable or obtained by any of the process described above.

Yet further, the present invention relates to the pharmaceutical compositions described above for use in the treatment of Hepatitis C viral infections.

Yet further, the present invention relates of any of the processes described above for the preparation of any of the compositions described above.

### EXPERIMENTAL

The present invention is further illustrated by the following examples.

### Reference Example 1: Determination of Physical Parameters

1.1 X-Ray Powder Diffraction (XRPD) Patterns

XRPD patterns were obtained with an X'Pert PRO diffractometer (PANalytical, Alme-lo, The Netherlands) equipped with a theta/theta coupled goniometer in transmission geometry, programmable XYZ stage with well plate holder, Cu-Kalpha1,2 radiation source (wavelength 0.15419 nm) with a focusing mirror, a 0.5° divergence slit, a 0.02° soller slit collimator and a 0.5° anti-scattering slit on the incident beam side, a 2 mm anti-scattering slit, a 0.02° soller slit collimator, a Ni-filter and a solid state PIXcel de-tector on the diffracted beam side. The diffractogram was recorded at room temperature at a tube voltage of 40 kV, tube current of 40 mA, applying a step size of 0.013° 2-theta with 40 sec per step in the angular range of 2° to 40° 2-theta. A typical precision of the 2-theta values is in the range of +0.2° 2-theta. Thus, a diffraction peak that appears for example at 8.1° 2-theta can appear between 7.9 and 8.3° 2-theta on most X-ray diffractometers under standard conditions.

### Comparative Example 1—Preparation of an Amorphous Solid Dispersion of the Compound of Formula (I) (Ledipasvir)

1.1 g Ledipasvir acetone solvate (crystalline Form II) prepared according to WO2013/184702 and 1.0 g Copovidone (Kollidon V64, BASF) were dissolved in 10 g ethanol and the resulting solution was evaporated to dryness in a rotary evaporator at 40° C. and 900-20 mbar. The resulting solid was grinded and dried at 60° C. for 15 hours. XRPD analysis of the solid confirmed the presence of the compound of formula (I) in amorphous form. The XRPD pattern is shown in FIG. 1.

### Comparative Example 2—Preparation of a Crystalline Solid Dispersion of the Compound of Formula (I) (Ledipasvir)

1.1 g Ledipasvir acetone solvate (crystalline Form II) prepared according to WO2013/184702 was suspended in a solution of 1.0 g Copovidone (Kollidon V64, BASF) in 3.0 g water and the solvent was evaporated to dryness in a rotary evaporator at 40° C. and 900-20 mbar. The resulting solid was grinded and dried at 60° C. for 16 hours. XRPD analysis of the solid confirmed the presence of the compound of formula (I) in crystalline Form II. The XRPD pattern is shown in FIG. 2.

### Comparative Example 3—Preparation of Compositions in the Form of a Tablet Comprising an Amorphous or a Crystalline Solid Dispersion of the Compound of Formula (I) (Ledipasvir) and the Compound of Formula (II) (Sofosbuvir) in Crystalline Form VII

Sofosbuvir of polymorphic Form VII was prepared by the process described above for example in 1.4. 2.0 g of polymorphic Form VII of the compound of formula (II) were blended with 900 mg of the solid dispersion of the compound of formula (I) prepared according to example 1 or to example 2, 0.825 g lactose, 0.4 g MCC 101, 125 mg crosscarmellose, 50 mg silicium dioxide and 37.5 mg magnesium stearate. For blending the compounds, an overhead reax mixture was used. The obtained mixture was compressed under a pressure of 10-25 kN to obtain flat tablets having a diameter of 10-25 mm. These tablets were crushed over a sieve having a mesh size of 0.5-1.5 mm. The obtained granulate was admixed with 500 mg MCC 101, 125 mg crosscarmellose and 37.5 mg magnesium stearate. For admixing the granulate with the excipients, an overhead reax mixture was used. The obtained mixture was compressed under a pressure of 10-25 kN to obtain 1000 mg oblong tablets of dimensions 18×8 mm. In particular, the tablets had the following compositions shown in Table 1, divided in intragranular and extragranular portions:

| | | |
|---|---|---|
| | content | content |
| Component | [mg] | [mg] |

Intragranular

| | | |
|---|---|---|
| Sofsobuvir polymorphic form VII | 400 | 400 |
| Ledipasvir solid dispersion | 180 | |
| according to example 1 | | |
| Ledipasvir solid dispersion | | 180 |
| according to example 2 | | |
| Lactose | 165 | 165 |
| MCC 101 | 80 | 80 |
| Crosscarmellose | 25 | 25 |
| Silicium dioxide | 10 | 10 |
| Magnesium stearate | 7.5 | 7.5 |

Extragranular

| | | |
|---|---|---|
| MCC 101 | 100 | 100 |
| Crosscarmellose | 25 | 25 |
| Magnesium stearate | 7.5 | 7.5 |
| Total | 1000 | 1000 |

### Comparative Example 4—Preparation of Compositions in the Form of a Tablet Comprising an Amorphous or a Crystalline Solid Dispersion of the Compound of Formula (I) (Ledipasvir) and the Compound of Formula (II) (Sofosbuvir) in Amorphous Form

Sofosbuvir of polymorphic Form I was prepared according to WO 2011/123645 A, Example 10. 2.0 g of polymorphic Form I of the compound of formula (II) were blended with 900 mg of the solid dispersion of the compound of formula (I) prepared according to example 1 or to example 2 and the mixture was molten. The solidified molten product was crushed over a sieve having a mesh size of 0.5-1.5 mm. The obtained granulate was admixed with 825 mg lactose, 900 mg MCC 101, 250 mg crosscarmellose, 50 mg silicon dioxide and 70 mg magnesium stearate. For admixing the granulate with the excipients, an overhead reax mixture was used. The obtained mixture was compressed under a pressure of 10-25 kN to obtain 1000 mg oblong tablets of dimensions 18×8 mm. In particular, the tablets had the following compositions shown in Table 2:

| | | |
|---|---|---|
| | content | content |
| Component | [mg] | [mg] |

Intragranular

| | | |
|---|---|---|
| Sofsobuvir I | 400 | 400 |
| Ledipasvir solid dispersion | 180 | |
| according to example 1 | | |
| Ledipasvir solid dispersion | | 180 |
| according to example 2 | | |
| Lactose | 165 | 165 |
| MCC 101 | 180 | 180 |
| Crosscarmellose | 50 | 50 |
| Silicondioxid | 10 | 10 |
| Magnesium stearate | 15 | 15 |
| Total | 1000 | 1000 |

### Comparative Example 5—Preparation of an Amorphous Solid Dispersion Comprising the Compound of Formula (I) (Ledipasvir) and the Compound of Formula (II) (Sofosbuvir)

Form I of sofosbuvir was prepared according to WO 2011/123645 A1, Example 10. 1.1 g Ledipasvir acetone solvate (crystalline Form II) prepared according to WO2013/184702, 4.4 g Sofosbuvir (Form I) and 1.0 g Copovidone (Kollidon V64, BASF) were dissolved in 16 g ethanol and the resulting solution was evaporated to dryness in a rotary evaporator at 40° C. and 900-20 mbar. The resulting solid was grinded and dried at 60° C. for 18 hours. XRPD analysis of the solid confirmed the presence of the compound of formula (I) and compound of formula (II) in amorphous form. The XRPD pattern is shown in FIG. 3.

### Comparative Example 6—Preparation of a Composition in the Form of a Tablet Comprising an Amorphous Solid Dispersion Comprising the Compound of Formula (I) (Ledipasvir) and the Compound of Formula (II) (Sofosbuvir)

2900 g of the amorphous solid dispersion of Ledipasvir and Sofosbuvir prepared according to example 5 were blended with 825 mg lactose, 400 mg MCC 101, 125 mg crosscarmellose, 50 mg silicon dioxide and 37.5 mg magnesium stearate. For blending the compounds, an overhead reax mixture was used. The obtained mixture was compressed under a pressure of 5-15 kN to obtain flat tablets having a diameter of 10-25 mm. These tablets were crushed over a sieve having a mesh size of 0.5-1.5 m. The obtained granulate was admixed with 500 mg MCC 101, 125 mg crosscarmellose and 37.5 mg magnesium stearate. For admixing the granulate with the excipients, an overhead reax mixture was used. The obtained mixture was compressed under a pressure of 10-25 kN to obtain 1000 mg oblong tablets of dimensions 18×8 mm. In particular, the tablets had the following compositions shown in Table 3, divided in intragranular and extragranular portions:

| | |
|---|---|
| | content |
| Component | [mg] |

Intragranular

| | |
|---|---|
| Ledipasvir and Sofosbuvir solid dispersion | 580 |
| according to Example 5 | |
| Lactose | 165 |
| MCC 101 | 80 |
| Crosscarmellose | 25 |
| Silicondioxid | 10 |
| Magnesium stearate | 7.5 |

Extragranular

| | |
|---|---|
| MCC 101 | 100 |
| Crosscarmellose | 25 |
| Magnesium stearate | 7.5 |
| Total | 1000 |

### Comparative Example 7—Preparation of a Composition in the Form of a Tablet Comprising an Amorphous Solid Dispersion Comprising the Compound of Formula (I) (Ledipasvir) and the Compound of Formula (II) (Sofosbuvir)

2900 mg of the amorphous solid dispersion of Ledipasvir and Sofosbuvir prepared according to example 5 were blended with 825 mg lactose, 900 MCC 101, 250 mg crosscarmellose, 50 mg silicon dioxide and 70 mg magnesium stearate. For admixing the granulate with the excipients, an overhead reax mixture was used. The obtained mixture was compressed under a pressure of 10-25 kN to obtain 1000 mg oblong tablets of dimensions 18×8 mm. In particular, the tablets had the following compositions shown in Table 4:

| | |
|---|---|
| | content |
| Component | [mg] |

Intragranular

| | |
|---|---|
| Ledipasvir and Sofosbuvir solid dispersion | 580 |
| according to Example 5 | |
| Lactose | 165 |
| MCC 101 | 180 |
| Crosscarmellose | 50 |
| Silicondioxid | 10 |
| Magnesium stearate | 15 |
| Total | 1000 |

### Comparative Example 8—Composition Comprising the Crystalline Compound of Formula I (Ledipasvir) for Dissolution Studies

| | content |
|---|---|
| Component | [mg] |
| Crystalline Ledipasvir acetone solvate | 200 |
| Polysorbat 80 | 100 |

200 mg crystalline Ledipasvir acetone solvate (crystalline Form II) prepared according to WO2013/184702 and 100 mg Polysorbat 80 were blended in a mortar with a pistil until a homogenous mixture was achieved.

### Comparative Example 9—Dissolution Experiments

The dissolution profiles for Sofosbuvir and Ledipasvir in the compositions according to Comparative Examples 3a, 3b, 4b and 7 above were determined using a USP Type 2 dissolution apparatus in 900 ml 1,5% polysorbate 80 in 10 mM Sodium phosphate Puffer pH 6.0 as the medium with 75 rpm at 37° C. (i.e. using the same conditions described in WO2014/120981). In addition, the dissolution profile for a composition comprising only Ledipasvir (see Comparative Example 8) was also determined using the same conditions as described above.

As can be seen from FIGS. 4 and 5 below, under the tested experimental conditions the solubility of Ledipasvir alone is very low, resulting in less than 20% dissolution after 70 minutes. In contrast, using the compositions of the invention, the dissolution of both Sofosbuvir and Ledipasvir, and in particular of Ledipasvir, was very fast under the tested experimental conditions, resulting in an increased dissolution rate.

In particular, the tablet composition according to Comparative Example 7 wherein Sofosbuvir is in amorphous form and Ledipasvir is in amorphous form results in complete dissolution for both compounds after 70 minutes. This is comparable to the data reported in WO2014/120981, which reports dissolution data for compositions comprising crystalline Sofosbuvir and amorphous Ledipasvir. According to WO2014/120981 ([173]), the tablet formulations described therein display greater than 85% dissolution for both compounds after 30 minutes. The tablet composition according to Comparative Example 7 of the present invention shows >99% dissolution for Sofosbuvir after 30 minutes. For Ledipasvir, 74% dissolution is achieved after 30 minutes and 87% dissolution is reached after 40 minutes, which is comparable to the compositions of WO2014/120981. However, unlike the compositions of WO2014/120981, the tablet composition according to Comparative Example 7 of the present invention comprises both active compounds Sofosbuvir and Ledipasvir in an amorphous form. In addition to the advantageous dissolution rate, this composition is much easier to prepare, since both active compounds (i.e. the compounds of formula (I) Ledipasvir and of formula (II) Sofosbuvir) as well as suitable excipients (such as for example copovidone) can be dissolved in a single solvent system and then dried, leading to a composition comprising both compounds of formula (I) and of formula (II) which can be easily prepared in a single process step. Advantageously, only one solvent system is needed, which eliminates the use of further solvents or solvent mixtures, and the mixture can be dried employing a variety of methods such as described in Comparative Example 5 (such as for example solvent evaporation, spray-drying, lyophilization, melt extrusion and other similar methods known in the art).

Another preparation having the advantages described above (i.e.such as increased solubility and ease of preparation) is the use of a melt-extrusion process. Advantageously, the compound of formula (I) Ledipasvir either in amorphous or in crystalline form can be dissolved in a suitable solvent (such as for example ethanol) and admixed with the compound of formula (II) Sofosbuvir either in crystalline or amorphous form and with suitable excipients (such as for example copovidone). Subjecting the mixture to melt extrusion leads to a composition in which both active compounds are in amorphous form and in the form of a solid dispersion. Advantageously, only one solvent system is needed, which eliminates the use of further solvents or solvent mixtures.

Even more advantageously, the compound of formula (I) Ledipasvir in amorphous form can be admixed with the compound of formula (II) Sofosbuvir either in crystalline or amorphous form and with suitable excipients (such as for example copovidone) and the resulting mixture can be subjected to a melt extrusion process thus leading to a composition in which both active compounds are in amorphous form and in the form of a solid dispersion. In this case, no solvent is needed at all, resulting in a simple, unwasteful and efficient process.

The thus advantageously obtained compositions described above can then be easily formulated into a tablet, such as the tablet composition of Comparative Example 7.

### SHORT DESCRIPTION OF THE FIGURES

FIG. 1 shows a representative X-ray powder diffraction (XRPD) pattern of a solid dispersion comprising the compound of formula (I) in amorphous form according to Comparative Example 1 of the present invention, as determined according to Reference Example 1.1. The x-axis shows the 2-theta angle/°, with tick marks, from left to right, at 10, 20, 30° 2-theta. The y-axis shows the intensity/counts, with tick marks, from bottom to top, at 200, 400, 600, 800, 1000, 1200, 1400.

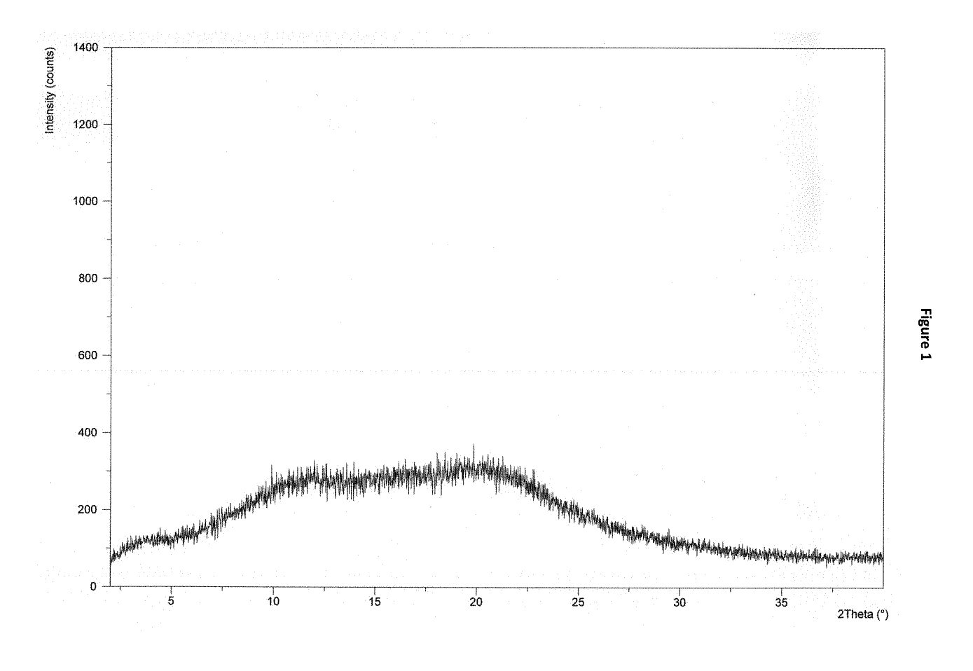

FIG. 2 shows a representative X-ray powder diffraction (XRPD) pattern of a solid dispersion comprising the compound of formula (I) in crystalline Form II according to Comparative Example 2 of the present invention, as determined according to Reference Example 1.1. The x-axis shows the 2-theta angle/°, with tick marks, from left to right, at 10, 20, 30° 2-theta. The y-axis shows the intensity/counts, with tick marks, from bottom to top, at 200, 400, 600, 800, 1000, 1200, 1400.

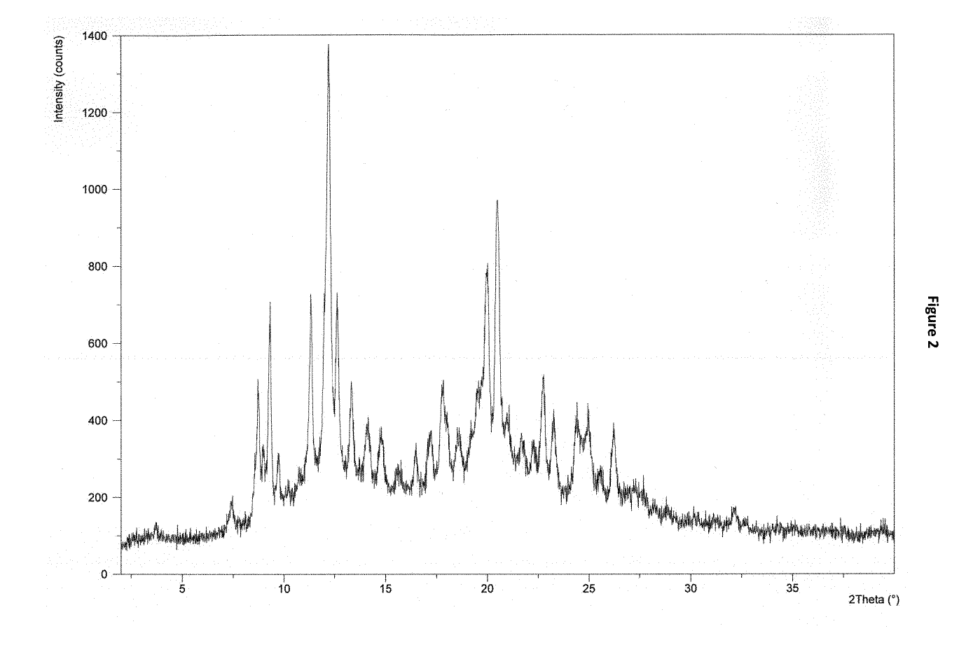

FIG. 3 shows a representative X-ray powder diffraction (XRPD) pattern of a solid dispersion comprising the compound of formula (I) and the compound of formula (II) in amorphous form according to Comparative Example 5 of the present invention, as determined according to Reference Example 1.1. The x-axis shows the 2-theta angle/°, with tick marks, from left to right, at 10, 20, 30° 2-theta. The y-axis shows the intensity/counts, with tick marks, from bottom to top, at 200, 400, 600, 800, 1000, 1200, 1400.

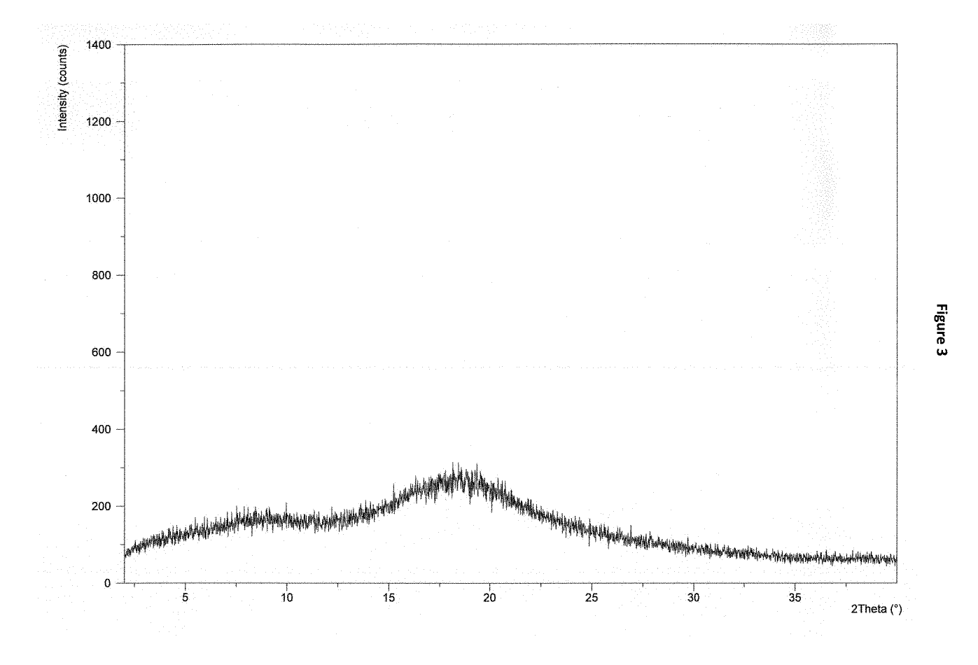

FIG. 4 shows the dissolution profile of the compound of formula I Ledipasvir for the compositions according to Comparative Examples 3a, 3b, 4b, 7 and 8. The x-axis shows the time in minutes and the y-axis shows the % of dissolved compound of formula I.

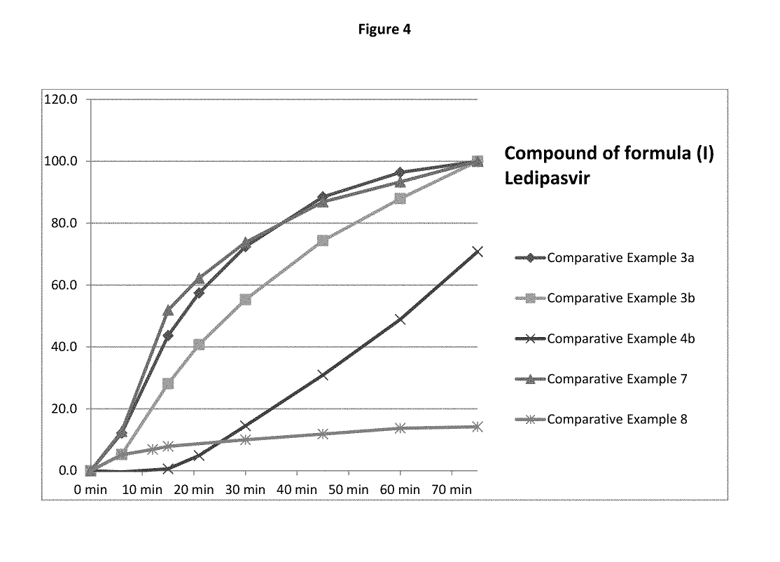

FIG. 5 shows the dissolution profile of the compound of formula II Sofosbuvir for the compositions according to Comparative Examples 3a, 3b, 4b and 7. The x-axis shows the time in minutes and the y-axis shows the % of dissolved compound of formula II.

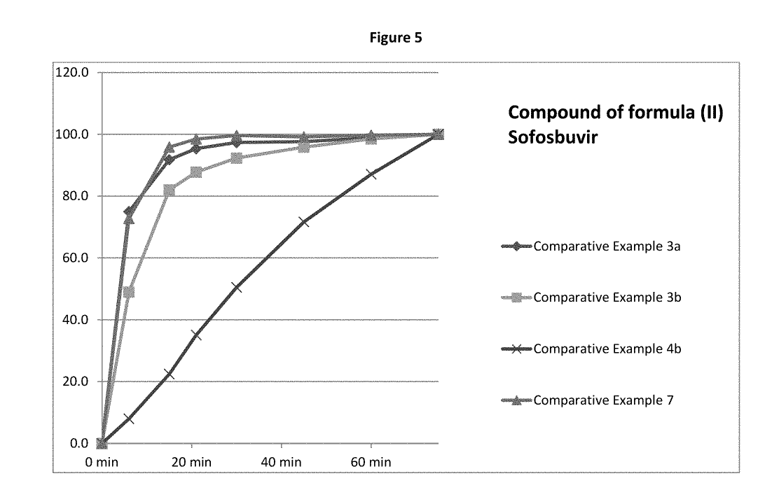

1. A pharmaceutical composition comprising a compound of formula (I) 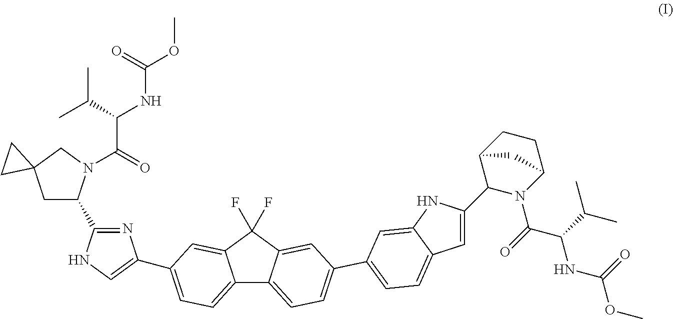 [SMILES: COC(=O)N[C@H](C(=O)N1CC2(CC2)C[C@H]1c1nc(-c2ccc3c(c2)C(F)(F)c2cc(-c4ccc5cc(C6[C@H]7CC[C@H](C7)N6C(=O)[C@@H](NC(=O)OC)C(C)C)[nH]c5c4)ccc2-3)c[nH]1)C(C)C]
or a pharmaceutically acceptable salt or solvate thereof and a compound of formula (II) 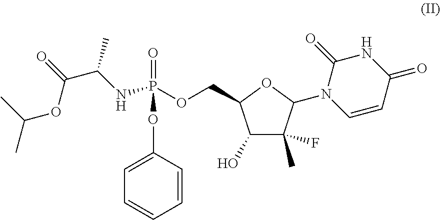 [SMILES: CC(C)OC(=O)[C@H](C)N[P@](=O)(OC[C@H]1OC(n2ccc(=O)[nH]c2=O)[C@](C)(F)[C@@H]1O)Oc1ccccc1]
or a pharmaceutically acceptable salt or solvate thereof, wherein the compound of formula (I) and the compound of formula (II) are in amorphous form.

2. The pharmaceutical composition of claim 1, wherein the compound of formula (I) is a solid dispersion.

3. The pharmaceutical composition of claim 1, wherein the compound of formula (II) is a solid dispersion.

4. The pharmaceutical composition of claim 1, wherein the compound of formula (I) and the compound of formula (II) are a homogeneous solid dispersion.

5. The pharmaceutical composition of claim 1 further comprising at least one pharmaceutically acceptable excipient.

6. The pharmaceutical composition of claim 5, wherein the at least one pharmaceutically acceptable excipient is a pharmaceutically acceptable polymer.

7. The pharmaceutical composition of claim 6, wherein the at least one pharmaceutically acceptable polymer is selected from the group consisting of hypromellose, copovidone and povidone.

8. The pharmaceutical composition of claim 1 in the form of a tablet.

9. A tablet composition comprising the pharmaceutical composition of claim 1.

10. The tablet composition of claim 9, further comprising at least one pharmaceutically acceptable excipient.

11. The pharmaceutical composition of claim 5, wherein the at least one pharmaceutically acceptable excipient further comprises at least one diluent, or at least one disintegrant, or at least one glidant, or at least one lubricant, or a combination of two or more thereof.

12. A process for the preparation of a pharmaceutical composition comprising a compound of formula (I) 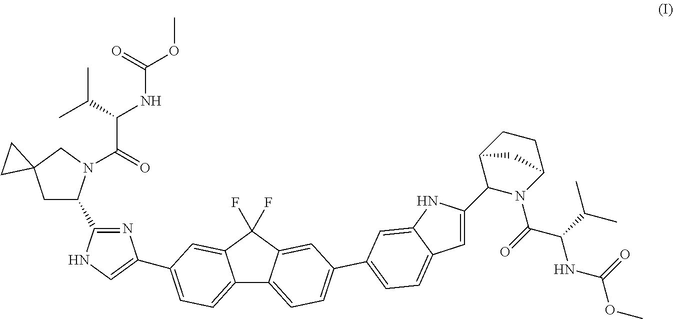 [SMILES: COC(=O)N[C@H](C(=O)N1CC2(CC2)C[C@H]1c1nc(-c2ccc3c(c2)C(F)(F)c2cc(-c4ccc5cc(C6[C@H]7CC[C@H](C7)N6C(=O)[C@@H](NC(=O)OC)C(C)C)[nH]c5c4)ccc2-3)c[nH]1)C(C)C]
or a pharmaceutically acceptable salt or solvate thereof and a compound of formula (II) 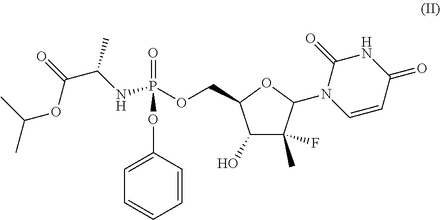 [SMILES: CC(C)OC(=O)[C@H](C)N[P@](=O)(OC[C@H]1OC(n2ccc(=O)[nH]c2=O)[C@](C)(F)[C@@H]1O)Oc1ccccc1]
or a pharmaceutically acceptable salt or solvate thereof, wherein the compound of formula (I) and the compound of formula (II) are in amorphous form, the process comprising the steps of:
(i) providing a compound of formula (I) or a pharmaceutically acceptable solvate or salt thereof,
(ii) providing a compound of formula (II) or a pharmaceutically acceptable salt thereof,
(iii) mixing the compound of formula (I) or a pharmaceutically acceptable solvate or salt thereof and the compound of formula (II) or a pharmaceutically acceptable salt thereof,
(iv) optionally blending the mixture provided in (iii) with at least one pharmaceutically acceptable excipient, and
(v) optionally preparing a tablet based on the blend obtained in (iv).

13. The process of claim 12, wherein (iii) comprises
(iii.1) adding a suitable amount of a pharmaceutically acceptable matrix to the compound of formula (I) and to the compound of formula (II) provided in (i) and (ii),
(iii.2) adding a suitable amount of at least one suitable solvent,
(iii.3) removing the at least one solvent, and
(iii.4) optionally milling and drying the solid resulting from (iii.3)

14. The process of claim 13, wherein the pharmaceutically acceptable matrix is a pharmaceutically acceptable polymer.

15. The process of any of claim 13, wherein the at least one suitable solvent is ethanol.
